SEQUENCE LISTING (29 sequences)

SEQ ID NO: 1 - DNA, 18 bp, Artificial
  Reverse primer (ActinS) used in comparative PCR experiment.
gagacaacattggcatgg

SEQ ID NO: 2 - DNA, 25 bp, Artificial
  Oligonucleotide utilized in reverse transcription reaction of beta-actin transcripts.
acagcacactttgtagagacctggg

SEQ ID NO: 3 - DNA, 34 bp, Artificial
  Exemplary stem-loop chimeric oligonucleotide designed to have a stable stem-loop secondary structure under transcription reaction conditions.
tctacaaagacagcacactttgtagagacctggg

SEQ ID NO: 4 - DNA, 19 bp, Artificial
  Exemplary forward hemi-nested primer utilized in comparative PCT experiment.
agcacactttgtagagacc

SEQ ID NO: 5 - DNA, 30 bp, Artificial
  Exemplary stem-loop chimeric RT oligonucleotides (SCRO) made in accordance with the teachings of the invention.
tcaccgttccccgccgtcggtgggcgctac

SEQ ID NO: 6 - DNA, 19 bp, Artificial
  Exemplary basis for designing exemplary PCR primer, based on Den 2 genome.
tgaaacgcgagagaaaccg

SEQ ID NO: 7 - DNA, 12 bp, Artificial
  Intermediate sequence based on SEQ.ID.NO.6 used for exemplary primer design.
tgaaacgcgaga

SEQ ID NO: 8 - DNA, 10 bp, Artificial
  Sequence based upon SEQ.ID.NO.7 used in exemplary primer design.
tgaaacgcga

SEQ ID NO: 9 - DNA, 13 bp, Artificial
  DNA sequence of SEQ.ID.NO.8 having GAA added to the 3′ end, thus raising the Tm.
tgaaacgcgagaa

SEQ ID NO: 10 - DNA, 17 bp, Artificial
  Exemplary hemi-nested PCR primer having 3′ protruding portion and 4 Gs added to the 5′ end of the sequence provided in SEQ.ID.NO.9.
ggggtgaaacgcgagaa

SEQ ID NO: 11 - DNA, 14 bp, Artificial
  Exemplary SCRO sequence.
ggggtgaaacgcga

SEQ ID NO: 12 - DNA, 6 bp, Artificial
  Deleted portion of SEQ.ID.NO.5 providing another exemplary convertible oligonucleotide SEQ.ID.NO.28.
tcaccg

SEQ ID NO: 13 - DNA, 18 bp, Artificial
  Forward primer for amplifying Dengue 2 NS2A region.
ggacatgggcagattgac

SEQ ID NO: 14 - DNA, 18 bp, Artificial
  Reverse primer for amplifying Dengue 2 NS2A region.
tccttttcttgttggttc

SEQ ID NO: 15 - DNA, 21 bp, Artificial
  Forward primer directed to envelope region of Dengue genome.
aggatggggaaatggatgtgg

SEQ ID NO: 16 - DNA, 21 bp, Artificial
  Reverse primer directed to envelope region of Dengue genome.
ttctgtggcccctgtgagtgc

SEQ ID NO: 17 - DNA, 24 bp, Artificial
  Forward primer to NS2A region of Dengue genomic RNA.
acctgggaagagtgatggttatgg

SEQ ID NO: 18 - DNA, 24 bp, Artificial
  Reverse primer to NS2A region of Dengue genomic RNA.
atggtctctggtatggtgctctgg

SEQ ID NO: 19 - DNA, 18 bp, Artificial
  Exemplary hemi-nested strand-specific PCR primer.
cgttccccgccgtcggtg

SEQ ID NO: 20 - DNA, 18 bp, Artificial
  Exemplary hemi-nested strand-specific PCR primer.
tcactgcatttgggacgc

SEQ ID NO: 21 - DNA, 20 bp, Artificial
  Forward primer to actin transcript.
acaacggctccggcatgtgc

SEQ ID NO: 22 - DNA, 20 bp, Artificial
  Reverse primer to actin transcript.
ggtcatcttttcacggttgg

SEQ ID NO: 23 - DNA, 12 bp, Artificial
  Portion of SEQ.ID.NO.5 complementary to the negative replicative strand of Dengue.
ggtgggcgctac

SEQ ID NO: 24 - DNA, 11 bp, Artificial
  Portion of another exemplary SCRO, complementary to the positive replicative strand of RSV.
cacggtgacac

SEQ ID NO: 25 - DNA, 21 bp, Artificial
  RSV MP2-specific sense primer.
ctcttggtatagttggagtgc

SEQ ID NO: 26 - DNA, 21 bp, Artificial
  RSV antisense primer.
tcaccgttccccgccgtccac

SEQ ID NO: 27 - DNA, 21 bp, Artificial
  RSV MP2-specific antisense primer.
ttggagaaattgttgagtggc

SEQ ID NO: 28 - DNA, 24 bp, Artificial
  Exemplary SCRO based upon DNS-1 having stem- loop portion of DNS-1 deleted.
ttccccgccgtcggtgggcgctac

SEQ ID NO: 29 - DNA, 30 bp, Artificial
  RSV RT1 primer designed in accordance with thermodynamic and nucleotide selection parameters taught by the invention.
tcaccgttccccgccgtccacggttgacac

(19) United States
(12) Patent Application Publication
(10) Pub. No.: US 2006/0252040 A1
(43) Pub. Date: Nov. 9, 2006

(54) Strand specific detection and quantification

(76) Inventors: Azlinda Anwar, Kent Vale (SG); J. Thomas August, Baltimore, MD (US); Heng-Phon Too, Kent Vale (SG)

Correspondence Address: GREENBERG TRAURIG LLP, 2450 COLORADO AVENUE, SUITE 400E, SANTA MONICA, CA, 90404, US

(21) Appl. No.: 10/526,816

(22) PCT Filed: Sep. 4, 2003
(86) PCT No.: PCT/SG03/00209

§ 371 Date: Dec. 27, 2005

Related U.S. Application Data
(60) Provisional application No. 60/408,818, filed on Sep. 6, 2002.

(51) Int. Cl. C12Q 1/68; C12P 19/34

(52) U.S. Cl. 435/6; 435/91.2

(57) ABSTRACT

An aspect of the present invention provides for specific compositions and methods of use make possible accurate, simple and efficient detection and quantification of desired nucleic acid strands. Teachings of the present invention also provide for novel and efficient primer design for annealing to specific target nucleic acid strands, as well as for designing particularly accurate amplification reaction primers for amplification of desired nucleic acid strands.

### RELATED APPLICATIONS

This application is related and claims priority to U.S. Provisional Application Ser. No. 60/408,818, filed Sep. 6, 2002 and entitled “Quantification of Replicative Dengue Virus”, herein incorporated by reference in its entirety.

### FIELD OF THE INVENTION

The present invention generally relates to molecular biological arts. More particularly, the present invention relates to compositions and associated methods for distinguishing, detecting and quantifying specific strands of nucleic acids, including RNA strands.

### BACKGROUND OF THE INVENTION

Recent advances in molecular biology have led to the development of various isothermal (nucleic acid sequenced based amplification, rolling circle amplification, branched chain DNA amplification) and thermal (polymerase chain reaction and ligase chain reaction) amplification methods for the detection of nucleic acids. These methods have been successfully used to detect and identify pathogens. An improvement over conventional PCR is the recent development of fluorogenic Quantitative Real Time PCR (Qrt-PCR). All amplification methods for detecting and quantifying nucleic acids potentially suffer from one important limitation, the inability to distinguish between the amplification resulting from replicating viral RNA and the viral genome. These amplification methods thus detect nucleic acids regardless of the state (live or dead) of the organisms, rendering a result which is diagnostically correct as positive, but clinically as false-positive. Such a limitation restricts the use of these methods for identifying the site of viral replication (reservoir) and hence, studies on virus-cell tropism. The ability to detect and rapidly quantify replicating viruses is crucial in elucidating the pathogenesis of many viral diseases and is also useful in the evaluation of drugs designed to inhibit viral replication.

Recent attempts to detect replicative forms of RNA viruses using conventional reverse transcription-PCR (RT-PCR) have met with some success (Lanford et al., 1994; Gunji et al., 1994; Mizutani et al., 1996; Lerat et al., 1996; Laskus et al., 1997; Liu et al., 1997; Mellor et al., 1998; Seipp et al., 1998; Craggs et al., 2001; Lin et al., 2002; Wang et al., 2002; Vaughan et al., 2002; Navas et al., 2002). However, some of these methods required post PCR manipulations, such as Southern blotting (Lanford et al., 1994; Laskus et al., 1997; Liu et al., 1997; Seipp et al., 1998; Navas et al., 2002), to achieve specificity in detection. Accordingly, prior art methodologies are not amenable to development of or incorporation into high throughput screens, and only allow detection, not quantification, of replicative RNA. As a result, the contribution of replicative viral load to the pathogenesis of viral diseases cannot be determined.

Other reported methods utilize tagged reverse transcription (RT) primers and hot-start in conventional RT-PCR (Seipp et al., 1998; Craggs et al., 2001; Navas et al., 2002; Lin et al., 2002). While these studies reported amplification of the replicative RNA strand, specificity, as before, remained a challenge. Specificity of amplification was improved by initially performing reverse transcription at 70° C, in combination with two rounds of nested PCR amplification (Craggs et al., 2001). However, even with these extensive modifications, this strategy suffers from an important drawback, namely mispriming of tagged RT primer at high concentrations of the incorrect positive strand RNA (>10⁴ copies).

As an example of a typical prior art method to detect replicative viral nucleic acid, reverse transcription (RT) is first carried out using oligonucleotides complementary to the replicative strand of the target (same polarity as the genomic sequences). An example of the replicative process is shown in FIG. 1, where the genome is positive stranded. The cDNA produced is then amplified and detected by conventional prior art methods such as PCR/Southern blot analyses or quantified by Qrt-PCR. Two approaches based on this prior art strategy have been reported. One utilizes viral genome specific oligonucleotides as RT primers and the other utilizes chimeric oligonucleotides as RT primers.

Dengue Fever (DF) and Dengue Hemorrhagic Fever (DHF) have emerged as an important vector-borne viral disease of human beings during the last 25 years. The increasing spread of the Dengue virus and its associated high morbidity to humans inhabiting urban communities in the tropics and subtropics is a major concern. Since an estimated 2.5 billion people live in areas at risk of Dengue virus transmission, both DF and DHF increasingly pose as important health problems in these regions (Gubler, 2002; Guzman et al., 2002).

The Dengue virus, just one example of a single-stranded positive sense RNA virus, encodes a single polypeptide precursor that is subsequently processed into three structural (C, preM and E) and seven non-structural (NS) proteins in the endoplasmic reticulum (Chambers et al., 1990). Upon infection, both the negative and positive RNA strands are synthesized de novo, and virions are encapsidated. Three different forms of viral RNA are synthesized during the replication process: a positive strand RNA, a duplex-stranded replicative form (RF) RNA and a replicative intermediate (RI) RNA, the latter two of which contain negative strand viral RNA (Chambers et al., 2002; Chu et al., 1985; Cleaves et al., 1981).

Currently, typical methods that are utilized for diagnosing Dengue virus infections include serological detection of Dengue-specific antibodies, Dengue virus isolation and identification from virus-infected mosquito cell lines (Vomdam et al., 1997). These methods are sufficient for epidemiological purposes, allowing diagnosis, but have little impact upon patient management (Isturis et al., 2000). The ability to diagnose Dengue infection at the acute phase of illness would provide a means for differential diagnosis and enhance patient care management.

Recent advances in molecular biology have led to the development of various amplification methods for the identification of Dengue viruses (Deubel, 1997). Using primers complementary to common sequences present in Dengue RNA genomes, specific PCR-based tests have been developed to enable diagnosis and serotyping of the virus (Lanciotti et al., 1992). Recent reports on the use of fluorogenic RT-PCR has significantly extended the reproducibility, sensitivity and quantification of Dengue viruses (Callahan et al., 2001; Houng et al., 2000; Houng et al., 2001; Laue et al., 1999; Warrilow et al., 2002).

However the above-mentioned PCR based studies suffer an important limitation, as mentioned previously, namely the inability to distinguish the amplification of viral RNA from replicating and inactive virus, a crucial aspect for identifying Dengue virus-cell tropism. Consequently, there is a desideratum for compositions and methods enabling accurate detection and quantification of particular nucleic acid strands, such as viral nucleic acid strands, including, replicating Dengue virus, for example. Additionally, the availability of a specific and quantitative assay of replicative RNA, for example, may prove useful for the evaluation of drugs designed to inhibit viral replication. The recent attempts discussed above, utilizing Southern blot and RT-PCR methodologies are labor-intensive (Liu et al., 1997) and non-quantitative (Vaughan et al., 2002).

### SUMMARY OF THE INVENTION

Now in accordance with one aspect of the invention, there has been found compositions and associated methods of providing and use of same for distingushing, detecting and quantifying specific strands of nucleic acids.

According to one embodiment of the invention, a highly specific, rapid and sensitive method to distinguish, detect and quantify the replicative negative strand of an actively replicating virus is provided. This method exclusively detects desired negative RNA strand using both in vitro transcribed RNAs and virus-infected cells, with a specificity of at least 105 fold difference over positive strand genomic RNA. In addition, the method allows for in vivo detection of an exemplary replicative negative RNA strand.

According to one embodiment of the invention, quantification of the replicative forms of exemplary viruses including Dengue, Respiratory Syncytial Virus (RSV) and West Nile virus are provided in accordance with the teachings of the present invention.

In accordance with one exemplary embodiment of the present invention, amplification and detection by Qrt-PCR is utilized. Adaptations of fundamental teachings of the invention can be applied to other amplification methods including, but not limited to, isothermal methods (NASBA, rolling circle, branched chain, etc).

In accordance with another aspect of the present invention, the high specificity of the disclosed methods is achieved at two levels: at a RT step by the use of a convertible oligonucleotide, which can include a chimeric stem-loop RT primer comprising target sequences, such as virus specific sequences, and unique sequences, folded into a loop structure with optimal energetics to enhance RT specificity; and at an amplification step, such as a PCR step, using specific nested PCR primers to a stem-loop chimeric RT oligonucleotides primer (SCRO). In particular embodiments, both positive (+) and negative (−) strand RNA viruses are utilized, that is, detected and quantifiable. The teachings of the present invention can be extended to any of the single-stranded RNA viruses, including HIV, regardless of the polarity of the genome, as well as to quantification of replicative states of any RNA viruses in vitro and in vivo.

In accordance with still another aspect of the present invention, the design and consideration of particular oligonucleotide thermodynamic characteristics, such as melting temperature profiles for a SCRO are determined such that the specificity of annealing of a SCRO to a specific target nucleic acid species is increased, while stem-loop structures of the SCRO also help to reduce mispriming, is also provided.

In accordance with another aspect of the invention, a method for strand specific amplification is provided that includes determining nucleic acid sequences of a target nucleic acid strand, designing a convertible oligonucleotide based, at least in part, on the target nucleic acid strand, conducting a transcription reaction utilizing the convertible oligonucleotide and the target nucleic acid strand to provide at least one resultant complementary strand, conducting an amplification reaction to amplify the at least one resultant complementary strand and analyzing the amplification reaction. In particular embodiments the designing step further comprises a step for conducting thermodynamic analysis of the convertible oligonucleotide to predict the secondary structure of the convertible oligonucleotide under reaction conditions of at least one of a transcription reaction and under a amplification reaction. In particular embodiments, the predicted secondary structure of the convertible oligonucleotide provides for at least a portion of the convertible oligonucleotide in a stem-loop conformation under reaction conditions of a transcription reaction.

In still other embodiments, the designing step further comprises a step of considering predicted secondary structures of at least a first portion and a second portion of a convertible oligonucleotide, under differing reaction conditions. Some embodiments provide for a designing step wherein nucleotides are selected to provide the convertible oligonucleotide with at least a step-loop portion and a portion for annealing to at least a portion of a target nucleic acid strand.

In particular embodiments at least one hemi-nested primer for use in a amplification reaction is provided. In some embodiments, at least one hemi-nested primer has a Ta that is substantially similar a Tm of the convertible oligonucleotide under amplification reaction conditions. The hemi-nested primer can have a 3′ portion with added nucleotides complementary to a transcription reaction product that is itself at least in part complementary to the target sequence.

In still other embodiments, the designing step includes designing the convertible oligonucleotide having nucleotides that are complementary to a target nucleic acid strand and non-complementary portions to the target nucleic acid strand, where the non-complementary portions form a first conformation structure under transcription reaction conditions and wherein the same non-complementary portions form a second conformation structure under the amplification reactions.

In some embodiments, the first conformation structure has at least a stem-loop portion.

In some embodiments, a convertible oligonucleotide is provided comprising a first self-annealing portion and a second portion complementary, at least in part, to a target nucleic acid sequence. In some instances, the first self annealing portion is in a stem-loop conformation under conditions of a first reaction and converts to a second substantially linear conformation under differing reaction conditions than the first reaction conditions. The differing reaction conditions can be at least transcription and amplification reaction conditions. The convertible oligonucleotide can have a 3′ (second) portion that anneals to a target nucleic acid sequences, the 3′ portion can from about 5 about 15 nucleotides or from about 8 to about 12 nucleotides. In some embodiments, the convertible oligonucleotide content of guanine, cytosine or combination of both is equal to or greater than about 50% of the total nucleotide composition of the convertible oligonucleotide. In particular embodiments, the first self-annealing portion has a ΔG of about ≦−0.5 kcal/mol under transcription reaction conditions.

In still other embodiments of the invention, a stem-loop chimeric oligonucleotide is provided comprising a first portion capable of forming a self-annealing stem-loop under a first set of reaction conditions, a second portion that maintains a substantially linear conformation under the same first set of reaction conditions and is capable of annealing to a target nucleic acid sequence on a particular target strand of nucleic acid. The stem-loop forming chimeric oligonucleotide can have a first portion capable of forming a self-annealing stem loop and a second portion, wherein the overall appropriate nucleotide sequences forms a substantially linear conformation at a second set of reaction conditions dissimilar to the first set of reaction conditions. The first set of reaction conditions can be transcription reaction conditions and the second set of reaction conditions can be amplification conditions.

Accordingly, teachings of the present invention can be extended to the quantification of replicative states of any RNA viruses in vitro and in vivo.

### BRIEF DESCRIPTION OF THE FIGURES

The foregoing aspects and many of the attendant advantages of this invention will become more readily appreciated as the same becomes better understood by reference to the following detailed description, when taken in conjunction with the accompanying figures, wherein:

FIG. 1 is a schematic depicting an exemplary prior art method for detecting replicative strands;

FIG. 2 is an exemplary schematic of a method for detecting and amplifying a target nucleic acid strand in accordance with the teachings of the present invention;

FIG. 3 is an exemplary schematic of one strategy to obtain unique tags and viral sequences and their combinations for RT PCR;

FIG. 4 depicts the formation of an exemplary stem loop in accordance with the teachings of the invention;

FIG. 5 is a schematic depicting the formation of a stem-loop tag having gene/strand specific portions and having exemplary physical properties;

FIG. 6 is an electrophoresis gel depicting results of experiments comparing the use of an exemplary stem loop RT oligo to an oligo having no predicted secondary structure at a RT stage;

FIG. 7 shows the predicted secondary structure of an exemplary convertible oligonucleotide, in accordance with the teachings of the present invention;

FIG. 8 is a schematic showing exemplary hemi-nested PCR primers comprising 3′ protruding sequences;

FIG. 9 depicts exemplary criteria for designing a PCR primer in accordance with the teachings of the present invention;

FIG. 10 shows the primary sequence and predicted secondary structure of an exemplary SCRO oligonucleotide;

FIG. 11 depicts another exemplary oligonucleotide forming a stem loop structure;

FIG. 12 depicts RT-PCR results of analysis of C6/36 cells infected with replicative or inactivated Dengue 2 virus;

FIG. 13A depicts exemplary specificity and sensitivity of detection of exemplary Dengue negative strand RNA, underlying bars indicating relative positions of the curves generated with negative and positive stranded RNA at differing dilutions;

FIG. 13B shows products from the experiment of FIG. 13A run on an electrophoresis gel, showing no predicted products from non-targeted (positive) strand;

FIG. 13C depicts the validation of the integrity the in vitro transcribed negative and positive strand RNAs by real-time quantitative PCR;

FIG. 13D depicts an electrophoresis gel containing in vitro transcribed negative and positive strand RNAs;

FIG. 14 is an electrophoresis gel containing exemplary amplification of reverse transcribed Dengue genomic RNA;

FIG. 15A is an electrophoresis gel depicting the kinetics of Dengue replicative RNA strand synthesis by RT-PCR;

FIG. 15B is a graph depicting ribavirin inhibition of Dengue viral replication;

FIG. 16 shows the correlation between quantification of repetitive Dengue RNA by real-time quantitative PCR using total RNA of BHK-21 cells infected with Dengue virus and plaque titration;

FIG. 17A depicts exemplary results of quantitative real-time PCR of replicative strand RNA from mouse brains inoculated with replicative or inactivated virus;

FIG. 17B is an electrophoresis gel showing amplification products from mice infected with replicative (lanes 3-5) and non-replicative (lanes 1-3) amplification products from mice brains inoculated with inactivated virus;

FIG. 18A is an “hot-start” amplification plot obtained with Dengue standards using DNS2-f and NS2A-minus-r primers, ten-fold serial dilutions of 0.1 fmoles/25 ul are detectable;

FIG. 18B depicts Ct values plotted against log of known amounts of standards;

FIG. 19 is depicts primary sequence and predicted secondary structure of another exemplary SCRO designed in accordance with the teachings of the present invention;

FIG. 20A depicts specificity and sensitivity of detection in accordance with the present invention for an exemplary positive strand RNA of an RSV virus utilizing primers and amplification methods in accordance with the invention;

FIG. 20B shows an electrophoresis gel validating the results shown in FIG. 20A;

FIG. 20C shows comparable Ct values for positive and negative strands of RSV, both primed with random hexamers;

FIG. 20D shows proper predicted sizes of random primed RSV RNA positive and negative strands;

FIG. 21 depicts the correlation between quantitation of replicative RSV RNA from total RNA from RSV infected Vero cells and plaque titration; and

FIG. 22 is an exemplary schematic of the order and use of various primers in accordance with the teachings of the invention.

### DETAILED DESCRIPTION OF THE PREFERRED EMBODIMENTS

Particular embodiments of the invention are described below in greater detail for the purpose for illustrating its principles and operation. However, various modifications may be made, and the scope of the invention is not limited to the exemplary embodiments or operations described. For example, while specific reference is made to negative strand detection and quantification, it can be appreciated that any specific nucleic acid strand may be detected and quantified in accordance with the teachings of the present invention. Likewise, the teachings of the present invention are accordingly applicable to transcription and amplification reactions alone or in combination, as recognizable to those of ordinary skill in the art.

Abbreviations utilized herein are as follows: Qrt-PCR quantitative real time PCR; RT, reverse transcription; Oligo, oligonucleotide; Tm, melting temperature; Ta, annealing temperature of PCR; nt, nucleotide; bp, base-pairs.

As an exemplary embodiment and to show proof of principle, exemplary positive (+) and negative (−) strand RNA viruses [Dengue (+), Respiratory Syncytial Virus (−)] are disclosed and utilized herein in accordance with the teachings of the invention. The methodologies and resultant compositions and uses are extendable to any single-stranded RNA viruses, including HIV, regardless of the polarity of the genomes. Qrt-PCR, a highly reliable method for quantifying nucleic acids, was exemplarily used for amplification purposes.

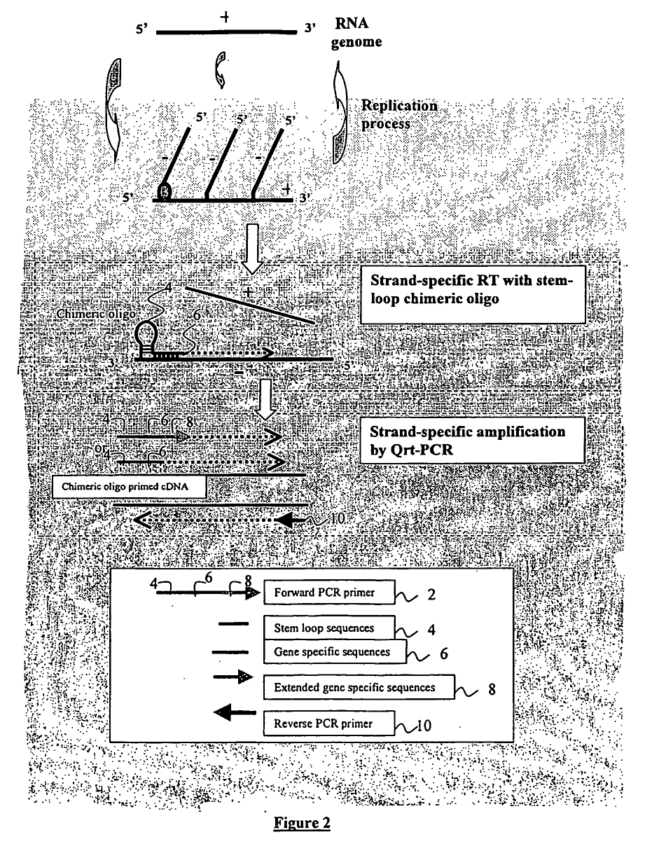

A general overview of steps involved in the detection and quantification of particular nucleic acid strands are described below (FIG. 2):

Step 1: Design of a specific convertible stem-loop chimeric oligonucleotide: Stem-loop chimeric oligonucleotide design is partially based upon the selection of unique target sequences having appropriate thermodynamics, as described below. Use of these optimal thermodynamic parameters provides for the enhancement of the RT reaction but does not affect downstream processes, e.g., PCR.

Step 2: Design of hemi-nested amplification primers: Use of specific primers for use in amplification reactions, which anneal to chimeric and gene specific sequences of resultant nucleic acid strands arising out RT reactions. The hemi-nested primer can extend beyond chimeric sequences to increase specificity at the amplification stage.

Multiplexing of specific targets can be realized by the utilization of florescence resonance energy transfer (FRET), one example of a detection methodology for multiplexing internal controls and targets, which provides for efficiency determination of RT-PCR. Probes such as, but not limited to, Beacon, Taqman, hybridization probes could also be utilized to detect amplification products provided in accordance with the teachings of the present invention. Extending this, if one part of an amplified sequence is detected with one FRET probe, another part of the sequence can be detected with another probe of different wavelength. If one of the sequences mutates (e.g., deleted), the other part can still be detected by the second probe.

The steps entailing replication of RNA viruses is well known to those of ordinary skill in the art. Viral RNA replication proceeds by first synthesizing a complementary sequence (replicative strand) from the viral RNA genome (in the case of Dengue, NS5, an RNA polymerase, is involved in the synthesis of the replicative strand). Subsequently, the genome is replicated by synthesizing complementary sequences to the replicative strand. This process is simply known as the replicative process (Chu et al, 1985).

In response to the lack of specificity and encumbrances associated with prior art methods, the teachings of the present invention provide for a novel method of selective identification and quantification of a desired nucleic acid strand by utilizing particularly designed convertible oligonucleotides, such as a SCRO, and hemi-nested PCR primers.

Prior art methods for designing oligonucleotides for use in various molecular biology protocols, such as reverse transcription for example, teach that primers should not self-anneal. This is a consideration that is programmed and utilized by various oligo-designing software such that self-annealing is avoided. In accordance with one aspect of the present invention and by utilizing the teachings provided herein, particular thermodynamic characteristics of the provided oligonucleotides, under selected reaction conditions, actually are selected/designed to encourage self annealing. In particular embodiments, this provides for stem-loop formation of at least a portion of the oligonucleotide and as a result, these stem-loop structures, having been designed to provide the desired thermodynamic characteristics (forming a stem-loop under transcription reaction conditions and linearizing under amplification reaction conditions, for example), can act as a highly selective (specific) priming site during amplification reactions while avoiding undesirable oligo-oligo interactions under transcription reaction conditions, detailed below.

In one embodiment, the detection and amplification of viral replicative RNA strand utilizing the stem loop and hemi-nested PCR method, in accordance with the teachings of the present invention, is exemplarily illustrated in FIG. 2. The specificity of the method described herein is achieved at the RT stage in conjunction with the specific amplification using the hemi-nested PCR primers. As shown in FIG. 2, the forward PCR primer 2 will not anneal to random hexamer primed cDNA, as such cDNA does not contain chimeric oligo sequences. A hemi-nested PCR primer is defined as an oligo sequence that anneals to the chimeric RT oligo at a PCR stage.

As discussed previously, to be able to quantify replicative viruses, methods utilized must provide an assay that is strand-specific, as replicating viruses synthesize complementary sequences to the genome prior to replication. Generally, the assay would have the following criteria:
1. Discriminates incorrect from correct strand.
   i) Strand specificity studies are preferably carried out with pure synthetic nucleic acids. This allows for determination of the dynamic range of the assay.
   ii) Strand specificity studies are conducted in the presence of random hexamer synthesized cDNA viral genome. As cellular nucleic acids may serve as random primers for RT (Gunji et al, 1994), the assay must NOT generate any product when amplification is conducted with cDNA synthesized with random primers. If product is generated, then strand-specificity is compromised.
   iii) Strand specificity studies should be carried out in the presence of heterogeneous nucleic acid mixtures consisting of both host (cellular) and viral RNA.
2. Large dynamic range of detection/quantification: Should have a dynamic range of about ≧10⁶ between the correct and incorrect strand.
3. High sensitivity: The assay should be able to detect 1-10 copies of the desired strand.
4. Desirable to be adaptable for high-throughput analyses: The assay should not have multiple steps or utilize detection methods that are labor intensive such as, for example, Southern blot analysis and multiple rounds of PCR (using nested primers), as taught by prior art methods.

The prior art methods utilizing gene specific oligos for both RT and PCR (for example, Laskus et al, 1997, Liu et al, 1997; Vaughn et al, 2002; Wang et al, 2002), and chimeric oligos with 5′ tag sequences (non-gene/target related sequences) for RT and subsequent amplification by PCR with primers directed at the tags (Lerat et al, 1996; Mellor et al, 1998; Siepps et al, 1998; Cragg et al, 2001; Navas et al, 2002; Lin et al, 2002) generally achieve low specificities (abilities to distinguish between the correct strand from the incorrect strand) (in the range of 10²-10⁵). Use of the same oligos for both RT and PCR result in a lack of strand specificity and also suffer from mispriming at the RT stage and subsequent amplification of nonspecific products.

Likewise, prior art usage of chimeric oligos (having a non-viral tag and viral sequences) for cDNA synthesis, with subsequent PCR amplification utilizing primers to the tag and a viral specific reverse primer, are also lacking. The primers utilized by these prior art methods do not have desirous Tm profiles, and thus do not form secondary structures (such as the stem loops taught by the present invention) and thus will result in misannealing and resultant misamplification.

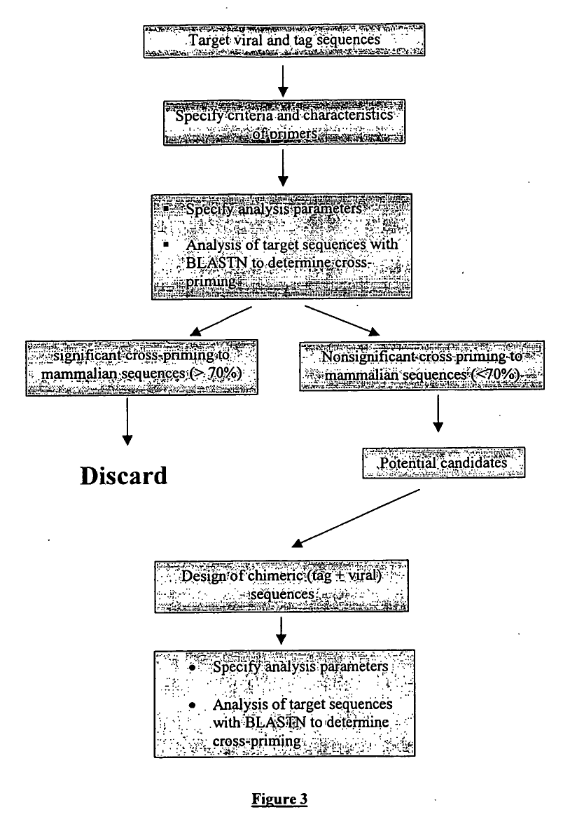

An exemplary schematic of one strategy for designing a convertible oligonucleotide, according to the teaching of the present invention, is depicted in FIG. 3. Exemplary steps and considerations for designing unique portions, such as tags, viral sequences and combinations therefrom, in order to provide the SCRO and hemi-nested PCR primers of the present invention is described below.

The rationale and strategies taught in accordance with the teachings of the present invention provide for the design of stem-loop chimeric RT oligos having convertible conformations and for hemi-nested PCR primers. Exemplary model viruses used to illustrate the use of this method in distinguishing and amplifying a selected nucleotide strand, such as a replicative RNA strand, are the Dengue and Respiratory Syncytial Viruses (RSV) (Table 1). The replication cycle of these viruses includes the de novo synthesis of negative (Dengue) or positive (RSV) RNA strand. The detection of these moieties is therefore indicative of the presence of actively replicating viruses.

TABLE 1
Exemplary Viruses

| | Genbank | Genome | |
|---|---|---|---|
| VIRUS | Accession No. | Strand | Target gene(s) |
| Dengue 2 NGC Strain | M29095; M19727 | + | NS genes |
| Respiratory synctial | AF013254 | − | MP2 gene |
| Virus B1 strain | | | |

The following characteristics/aspects are to be considered when designing an exemplary SCRO, here for use in replicating Dengue detection, for example, in accordance with the teachings of the invention.

Selection of a Stem-Loop Chimeric RT Oligo:
1. Optimal size of primer: (Note: For the exemplary cases utilized herein, Dengue and RSV, sequence homology to mammalian sequences is considered). Accordingly, a long primer sequence will lead to high homology with mammalian sequences (undesirable), yet a short primer size will have difficulty in specific priming to the target viral sequence. Thus, oligo lengths of about 5 to 15 nt, more preferably 8-12 nt are found to be optimal.
2. G+C content of primer: ≧50% to enable a relatively high annealing temperature at an amplification (such as PCR) stage.
3. Specificity of the 3′ sequence portion is targeted to viral (targeted) sequences.
4. Formation of a stem-loop structure having a tag sequence (chimera RT oligo).

In the exemplary case of the Dengue virus, analysis of overlapping short oligomers (12 mer in length) derived from target NS genes (total genome sizes of ˜10 kb) resulted in a few potential unique viral sequences, some of which are listed below. A majority of the oligomers analyzed showed homologies to extraneous mammalian sequences, rendering them unsuitable for use in this strategy (FIG. 3). Similar experiences were encountered in designing appropriate oligomers for the RSV.

Exemplary Unique Dengue Viral Sequences Having Low Homology With Extraneous Mammalian Sequences:

a) Dengue NS-l(DNS-1)

Length of domain: 1056 bp

No. bases scanned: 1056 bp

No. primers meeting criteria: 1; DNS-1 (Refer to enclosed copy of the DNS-1 sequence)

b) Dengue NS-2(DNS-2)

Length of domain: 1044 bp

No. bases scanned: 1044 bp

No. primers meeting criteria: 6; DNS2-1; DNS2-2 to DNS2-6

In selecting the viral (gene/target strand specific) segment of the SCRO to be assembled various considerations must be kept in mind, such as the length of the gene specific segment of the SCRO. An exemplary length, such as about 6-15 bp, more preferably about 8-12 bp or longer, can be utilized as long as the Tm of hybridization to the specific RNA is ±10° C. of the temperature used for RT. The reason for using a short oligo is that short oligos generally have low Tm comparable to the temperature used for RT and thus lengths in these ranges increase specificity of reverse transcription.

However, if the gene specific sequence of SCRO has a Tm similar to the temperature used for RT (42±5° C.), then this sequence alone will not be able to hybridize to the target cDNA at the annealing temperature of PCR (Ta). Thus, the gene specific segment of SCRO does not serve as a PCR primer at the Ta.

As an example, random hexamers with Tm well below the Ta can be used for RT and thus will not serve as amplification primers in PCR (consider a hexamer of G, this sequence will have a Tm<20° C.). This consideration allows RT to be performed at temperatures lower than PCR and yet when the hexamers are carried over significantly to the PCR stage, the hexamers will not be able to anneal to cDNA templates at the Ta.

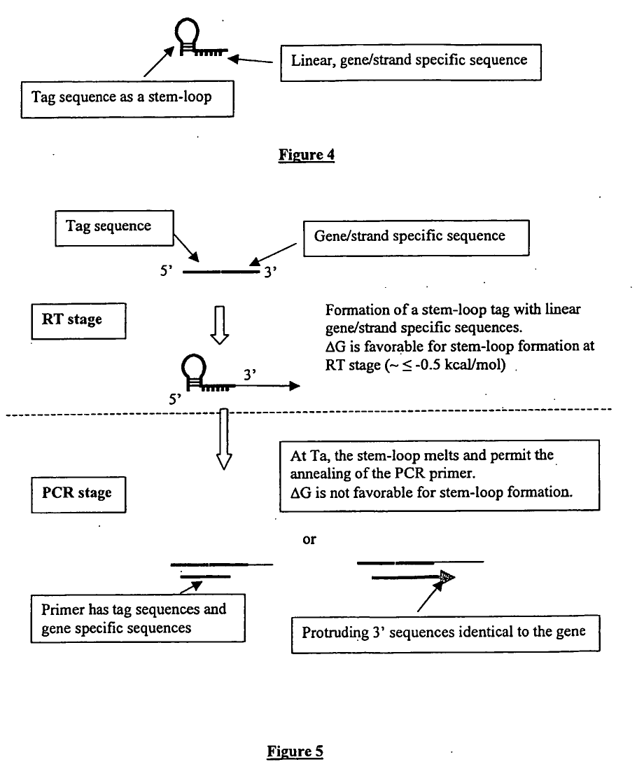

In one example, and in order for the SCRO of the present invention to serve as a template to which a PCR primer will anneal, extra sequences (tags) are added to the 5′ terminal. The resulting oligo is a chimeria of tag and gene specific/strand specific sequences which provides for a stem-loop (FIG. 4). The tag portion at the 5′ end of the oligo should not have sequences homologous to the gene of interest.

In order to avoid mispriming/misannealing to nonspecific targets at the RT stage, the choice of the tag is based on sequences that are preferably from non-related organisms. As an example, in the exemplary cases of the Dengue and RSV viruses exemplarily utilized herein, non-mammalian sequences were selected avoid mispriming at the RT-stage (FIGS. 2 and 3). Formation of a stem loop of the tag sequences reduces mispriming (FIG. 6), as is shown in the disclosed study utilizing beta-actin transcripts (below). The beta-actin was used as a tag deliberately. When compared to a non-structured oligo (no-loop-formation), the stem-loop oligo prevented mispriming to the beta-actin sequences even after 40 cycles of PCR.

In order to verify that utilization of a primer comprising a stem-loop segment, a primer structure counterintuitive to typical primer design, actually reduces mispriming, the following experiment was conducted.

A comparison was made between 2 chimeric RT oligos intentionally designed to misprime onto beta-actin transcripts. As beta-actin is abundantly expressed in liver, these 2 chimeric RT oligos (par2 and par2-zip9) were used for reverse transcription followed by PCR with the same reverse primer.

Accordingly, one microgram of total liver RNA was subjected to reverse transcription, (RT) with either 0.1 μM of Par2 or a Par2zip9 SCRO. RT was carried out at 42° C. using ImPromII (Promega, Madison, US) for 45 min, followed by 5 min of heat inactivation at 70° C. Twenty five percent of the cDNA synthesized were amplified (40 cycles of denaturation at 95° C. for 0.5 min, annealing at 60° C. for 1 min and extension at 72° C. for 1 min) with the hemi-nested PCR primer (NEST) and reverse primer (ActinS). Thirty percent of the products were then electrophoresed on 2% agarose and visualized by ethidium bromide (FIG. 6).

The sequences of the RT and PCR oligos are given below: underlined letters represent Dengue sequences, bold sequences prime to beta actin.

| | |
|---|---|
| ActinS: | |
| GAGACAACATTGGCATGG | (SEQ ID NO: 1) |
| Par2: | |
| ACAGCACACTTTGTAGAGACCTGGG | (SEQ ID NO: 2) |
| Par2zip9: | |
| TCTACAAAGACAGCACACTTTGTAGAGACCTGGG | (SEQ ID NO: 3) |
| NEST: | |
| AGCACACTTTGTAGAGACC | (SEQ ID NO: 4) |

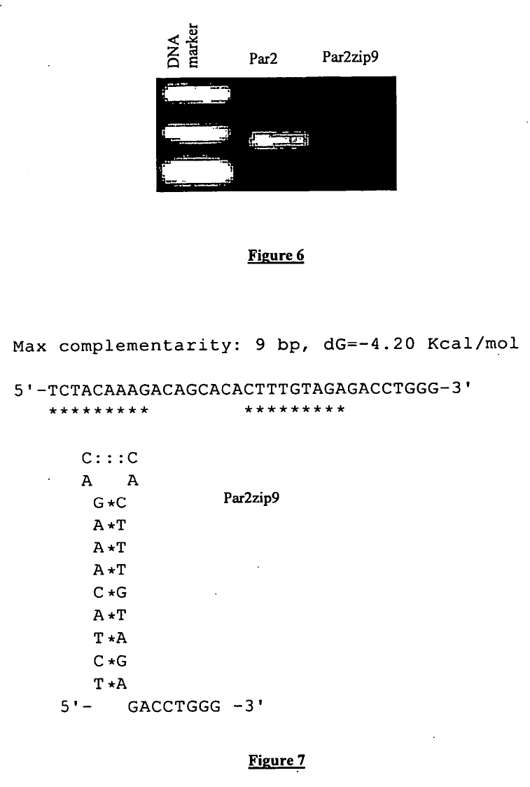

The predicted secondary structure of Par2zip9 is shown in FIG. 7 Par2 oligo does not form any stable secondary structures at the RT stage.

Results: Providing a stem-loop (Par2zip9) reduces amplification of beta actin as shown by the lack of amplified product in the Par2zip9 lane in FIG. 6. Thus, by forming secondary structure of the exogenous sequences at the 5′ end, it was possible to prevent annealing of the chimeric RT primer to actin. Conclusion: Stem-loop prevents priming to the 5′ sequences.

As discussed previously, an example of a template that was utilized in the design of an appropriate tag (stem-loop) sequence, in accordance with the teachings of the present invention, is the chalone synthase gene of Arabidopsis thaliana. In this example, tag sequences can be any sequences that do not anneal to mammalian sequences or the viral genome of interest. While the choice of using the Arabidopsis sequences (Reference sequence: chalone synthase gene of the Arabidopsis thaliana; Genbank accession: M86358) was because of the genetic differences between plants and mammals, other various divergent evolutionary relationships may also be utilized in order to consider particularly useful sequences (low homology), thus reducing the likelihood of mispriming.

In one aspect of an embodiment, as with the design of the viral oligos above, the criteria employed in the design of the tag (stem-loop) portion of the oligomer are, as discussed above:
1. Optimal size of tag and viral sequences to achieve optimal thermodynamics in PCR
2. G+C content of primer ≧50%
3. Formation of a stem-loop structure with the 5′ sequences (stem-loop chimera RT oligo)
4. The combined generated chimeric RT oligo comprising tag and viral sequences maintains specificity of annealing to the targeted viral sequence, with no significant cross priming to mammalian sequences.

Here, as in the analysis of the viral sequences, exemplary overlapping short oligomers (12 mer in length) derived from the chalone synthase gene (total size of ˜2 kb) resulted in 2 potential unique sequences that can be used as a tag in this strategy, listed below. The majority of the oligomers analyzed showed significant homologies to extraneous mammalian sequences (>70%), again rendering them unsuitable for use.

Chalone Sythase:
Length of domain: 1999 bp
No. bases scanned: 1999 bp
No. primers meeting criteria: 2
Result 1 (modified seq; 1668-1685)
Result 2 (691-708)

An aspect of the invention to keep in mind is that the stem-loop portion of the tag sequences should form favorably at the conditions during the RT stage and must also however, melt sufficiently for primers to anneal efficiently at the Ta of the amplification stage (Ex. PCR). If tag sequences do anneal to the undesired sequences, such as mammalian sequences, the tag's Tm must not be substantially similar to its Ta, otherwise the stem-loop portion of the SCRO may unfold due to hybridizations and result in misannealing to exogenous sequences. These characteristics may be investigated by a study of the thermodynamics (Gibbs free energy (ΔG)) of the stem-loop portion of the chimeric oligos.

ΔG can be considered, for a reaction, a direct measure of how far the reaction is from equilibrium. It is often described in the form: ΔG=ΔH-TΔS Where, ΔH=change in enthalpy; T=temperature of the reaction; and ΔS=change in entropy. If a reaction is exothermic (releases heat), ΔH will be negative (by convention). The reverse scenario (endothermic) results when, for example, NaCl (a highly structured crystal) dissolves, then ΔH will be in a positive values (the solution feels cool).

In the case of the overall reaction equation, the more positive the ΔG value is, the more energy is needed to push the reaction forward. The reverse (ΔG is more negative) results when the reaction is more likely to proceed on its own. According to the teachings of the invention, ΔG is calculated at the temperature of interest, for example, ΔG at 42 C for RT stage, etc.

Another way to express the propensity of a reaction to occur is to describe ΔG in terms of an equilibrium of a reaction. Consider the exemplary case of folded versus unfolded state of the exemplary SCRO, Dengue oligonucleotide DNS-1. In this example, the reaction can simply be described as:
DNS-1 (unfolded)⇄DNS-1 (folded) (1)

At equilibrium:
Keq=[DNS-1 (folded)]/[DNS-1 (unfolded)] (2)
Gibbs free energy can also be expressed with relation to the equilibrium of a reaction:
ΔG=RTlnKeq (3)

If there are more molecules of DNS-1 (folded) than DNS-1 (unfolded), then Keq will be >1. Substituting the Keq values into equation 3 will reveal the free energy to be negative. The more negative the ΔG is, the more spontaneous the reaction.

Accordingly, with the reaction described in terms of ΔG, an oligonucleotide can exist in two extreme forms (equation 1) and the equilibrium is described by Keq. In reality, there will be many states between the folded and the unfolded.

Hence, the oligonucleotide sequence (DNS-1) is unique (does not prime to known mammalian sequences) and has a more stable_structure (negative ΔG ) at RT conditions. The 5′ end of the oligo was designed to “zip” up (self-anneal) the unique and a part of gene-specific sequences so that mispriming is reduced. By forming a stable stem-loop structure, tag sequences will not be available for annealing to any sequences. A negative ΔG shows that stem-loop formation is favorable at the condition for RT. Hence, by so describing and utilizing Gibbs free energy (ΔG), the ability to design stable secondary structures (stem-loop) in accordance with the teachings of the present invention is provided. Prediction of secondary structures can be computed using the Zucker program found at (http://www.bio info.rpi.edu/applications/mfold), for example. Other programs that predict secondary structures may also be utilized.

Another aspect of the invention is the consideration of the Tm (melting temperatures) of the designed SCROs. Accordingly, Tm of the SCROs and hemi-nested PCR primers are determined by the exemplary calculations below. These are used as guides as these are predictive tools based on empirical data and provided useful guides for designing the various compositions utilized in the various embodiments presented herein.

Thermodynamic Tm: An exemplary method for calculating Tm is the nearest-neighbor thermodynamic values method (Breslauer et al., 1986). The formula for determining Tm is:

Tm=ΔH/(ΔS−Rln[DNA])−273.15+16.6*(log10[DNA]), where ΔH is the enthalpy, ΔS is the entropy, R is 1.987 cat K-1 mol-1, [DNA] is the DNA concentration, and Cs is the salt concentration.

Hybridization Tm: This method is generally used for DNA or RNA hybridization, especially in presence of high salt and formamide. It is more accurate with longer oligos (from about 15 to about 20 nucleotides). This Tm is calculated using the following formula:
For DNA:RNA hybridization: Tm=79.8+18.5*(log10[Na+])+0.58*[%(G+C)]+11.8*[%(G+C)]2−0.5*(% Formamide)−820/L−1.5 (% Mismatch);
where L is the length (bases) of the oligonucleotide and Na+ is the concentration of salt utilized during RT.

GC+AT Tm: the estimated Tm is the sum of the contribution of each base: 2° C. for A and T and 4° C. for G and C.

As mentioned previously, another aspect of the present invention relates designs and resultant characteristics therefrom of hemi-nested PCR primers utilized during amplification steps. Primers must anneal to the tag as well as the gene specific sequences of the SCRO (FIG. 5). As the gene specific segment of the SCRO may not have an appropriate Tm, the additional tag sequences will increase the Tm sufficiently to enable the PCR primer to anneal specifically, as mentioned above.

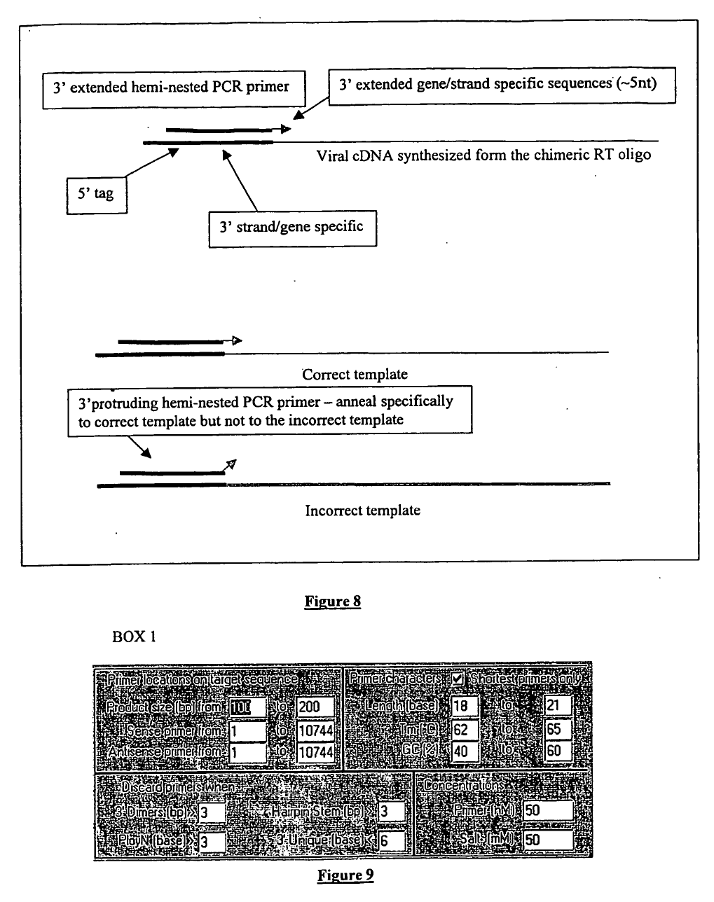

If the SCRO has homologous sequences to other genes (by chance), then extra 3′ nt(s), extending into the specified gene can be used to reduce nonspecific priming. Previously studies have shown that overlapping junctional sequences can discriminate between related transcripts with high specificity (10⁴ fold increase in specificity). The protruding sequences of the PCR primer can as short as, but not limited to, about 5 bp (FIG. 8).

As depicted in FIG. 5 the protruding PCR primer should have a Tm close to the Ta (±5° C.). It must not be able to anneal to cDNA synthesized using random primers. Thus, the gene specific segment of the SCRO (including the 3′ protruded nt) must not have Tm similar to Ta but less (<10° C.). The reverse primer employed is gene specific.

Therefore, and in accordance with one embodiment of the invention, guidelines for the design of chimeric stem-loop oligo and hemi-nested PCR primers for quantification of a particular nucleic acid strand, here exemplary shown as the negative strand of a RNA virus (Dengue virus) comprises:
(1) A SCRO having two segments: 5′ tag sequences that are unrelated to the viral sequence and a 3′ segment, for example about 8-12 nt long, is complementary to the desired viral sequences.
(2) The SCRO oligo sequence should not have significant homologies (about <70%) to known mammalian sequences in databases (eg., Genbank).
(3) The melting temperature (Tm) of the 3′ segment, when hybridized to the desired viral sequence, should be about ±7° C. of the temperature used for RT.
(4) The 3′ segment of the SCRO should not adopt any stable secondary structure at the condition used for RT (ΔG≧−0.5 kcal/mole).
(5) The 5′ tag sequence should adopt a stable secondary structure (stem-loop) at the condition used for RT (ΔG≦−0.5 kcal/mole).
(6) The loop should not anneal to known mammalian sequences. If the loop sequence is complementary to mammalian sequences, then the Tm of hybridization of the loop with the complement sequence must be less than the Tm of the stem portion.
(7) The free energy and Tm of the stem-loop may be calculated using the nearest-neighbor thermodynamic calculations and computed using the Zucker program, for example.
(8) At the PCR condition used, the chimeric RT oligo should not adopt any stable secondary structure to enable the efficient annealing of a hemi-nested PCR primer.

1(9) The hemi-nested PCR primer should hybridize to the target sequence with a Tm similar to the Ta (±10° C.).
(10) The sequence of the SCRO and the PCR primers should be designed so that there are no regions of complementarity which may increase background noise/signals.

The rationale for using the stem-loop structure is evident from the following:
(a) Thermodynamically stable stem-loop can restrain the tag at the 5′ end from misannealing to spurious, extraneous sequences.
(b) As reverse transcription is carried out at 42° C., for example, an optimal structure for the complementary viral but not to the tag sequence is required to anneal to the replicative viral strand of nucleic acids. This unique chimeric sequence is thus folded into a stem-loop structure at the 5′ end.
(c) The design of hemi-nested PCR primers enable the amplification of only the targeted replicative RNA reverse transcribed by the stem-loop chimeric RT oligo. The unfolding of the chimeric RT oligo must be energetically favored at the annealing temperature (Ta) to enable high efficiency of PCR (>70%).

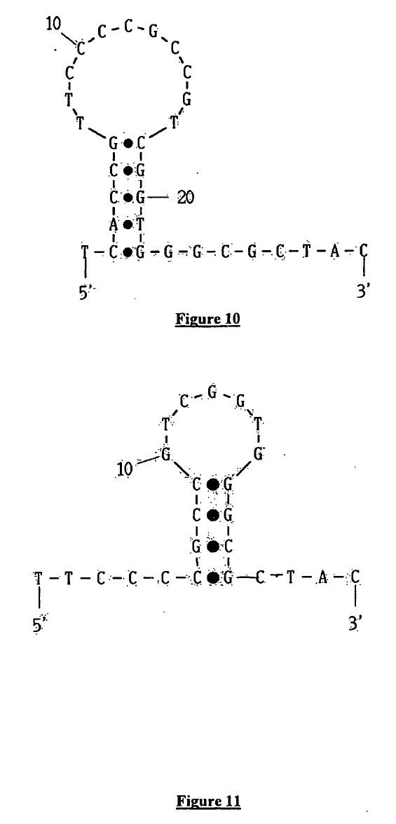

As an example a chimeric primer, (DNS-1), has the combined sequence and characteristics shown below (gap shown only to distinguish tag and viral portions (FIG. 10):

(SEQ ID NO: 5)

| | |
|---|---|
| 5′ TCA CCG TTC CCC GCC GTC | GGT GGG CGC TAC 3′ |
| Tag | Viral |

An exemplary BLAST search resulted in the finding of no significant mammalian sequences anneal to the 3′ end of the chimeric sequence (DNS-1). Since it was found that in this example this tag sequence does not anneal to known mammalian sequences, considerations of the Tm of loop sequences annealing to known mammalian sequences are not applicable in this instance.

The use of hemi-nested PCR primers with 3′ protruding sequences (FIG. 8) can be used to achieve high specificity without the use of prior art internal nested primers. The design of these primers is highly dependent on the properties of the stem-loop chimeric RT oligo and will be discussed below.

In FIG. 8, the hemi-nested PCR primer has 3′ sequences to a correct template and protrudes out of the chimeric RT-primer. As the annealing of the 3′ sequences is critical to the extension of the viral cDNA (here, having the SCRO incorporated due to the RT already taken place) by DNA polymerases, the misannealing at the 3′ end onto an incorrect template will not be efficiently extended.

The length of the 3′ protruding hemi-nested PCR primer, including the gene/strand specific sequences, will depend on the Tm of hybridization. If this segment has a Tm close to the Ta, then the primer can anneal to the template without the need to anneal to the cDNA synthesized with SCRO. Thus, this may prime to the incorrect cDNA templates and will also yield a product when amplifying with cDNA synthesized with random primers and thus is to be avoided.

Guideline for Designing 3′ protruding hemi-nested PCR primer:
1. Calculate Tm of the gene specific segment of SCRO plus protruding 3′ sequences. The Tm of the gene specific segment/sequence is about ≦10° C. of the Ta.
2. Subsequently, add tag sequences to this segment to have a Tm close to Ta, about ±10° C.

An exemplary PCR primer based on the criteria listed in Box 1 of FIG. 9 using the Den2 genome as a template, is shown. Assuming the SCRO has a gene specific sequence TGAAACGCGAGAGAAACCG (SEQ ID NO: 6), then it will have a Tm of ˜62° C. at the PCR stage. At the Ta of 60° C., the sequence will be optimal for annealing to the complementary template.

In the designing a gene/strand specific segment of SCRO, the sequence must have a Tm similar to the temperature used for RT, thus, this will reduce the sequence to about ≦12 bp (TGAAACGCGAGA (SEQ ID NO: 7) has a Tm of 43° C.). Assuming a 10 mer is designed (TGAAACGCGA) (SEQ ID NO: 8), the Tm is now about 35° C. This is suitable for RT but will not be optimal for PCR. If 3′ extra sequences are added, the overall sequence has a Tm<10° C. of the Ta. Thus, by adding extra 3 bases (TGAAACGCGAGAA (SEQ ID NO: 9)), the Tm of this sequence (44° C.) is still not optimal for PCR at 60° C. annealing. Therefore the Tm of the 3′ protruding hemi-nested PCR primer is increased by the addition of 5′ tag sequences until the overall Tm is about that of Ta.

An example of a sequence of the 3′ protruding hemi-nested PCR primer is: GGGGTGAAACGCGAGAA (SEQ ID NO: 10) Tm=61° C.

where GGGG is a part of the tag, TGAAACGCGA is the gene/strand specific sequence of SCRO, and GAA at the 3′ terminus is the protruding sequence. While GGGG is shown, any additional bp that bring the overall Tm to approximately the Ta will suffice (SCRO sequence is: GGGGTGAAACGCGA (SEQ ID NO: 11)) Tm of the gene specific strand is 35° C., suitable for RT but not for PCR.

Proof of concept of the use of extended protruding 3′ sequences to enable the discrimination of highly homologous sequences by Qrt-PCR, for example, is shown in recent papers (Wong & Too, 2000; Wong, Sia, Too, 2002; Too, 2003). In these works, it was shown that it was possible to distinguish specificity of amplifications of alternatively spliced isoforms of the GFR2 receptors. The strategy in these papers was to use primers which anneal identically to 3 isoforms at the 5′ terminal but differ by 5 nt at the 3′ terminal (the adjacent junction). The results showed that the use of such protruding (overlapping) primers enabled the specific amplifications of each isoform by >10⁴ fold. Thus, this strategy can be adapted to the design of the hemi-nested PCR primers as taught by the present invention.

An example of a SCRO designed and provided in accordance with the teachings of the present invention is depicted in FIG. 10. Various exemplary physiochemical properties of this stem-loop chimeric RT oligo, DNS-1, are as follows:

Calculated ΔG of the hairpin loop at the RT step=−2.9 kcal/mol

Tm (melting temperature) at RT step=62° C.

Na⁺=75 mM

Mg²⁺=3 mM

RT temperature=42° C.

Calculated ΔG of the hairpin loop at the PCR step 0.4 kcal/mol

Tm of the hairpin loop at PCR=64° C.

Na⁺=50 mM

Mg²⁺=2.5 mM

Annealing temperature=60° C.

Hence, at these exemplary salt and temperature conditions used for RT, the formation of a stem-loop structure is highly favoured. However, with the conditions used for PCR, with an annealing temperature of 60° C. used in this study, the formation of the stem-loop was less favorable, thus allowing sufficient relaxation and linearity of the incorporated SCRO portions to provide efficient annealing of the hemi nested PCR primers to the unique sequences for specific amplification.

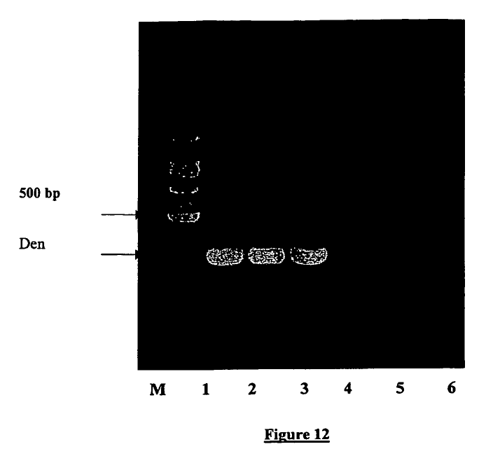

If the stem loop portion of DNS-l is deleted (in this case the stem sequence of 5′ TCACCG 3′ (SEQ ID NO: 12)), another exemplary stem loop structure of about the same energetics will be formed (mod-DNS-1, SEQ ID NO: 28), as depicted in FIG. 11. Specific sequences at the 3′ end are available for annealing to Dengue sequences.

An example of the strategy for designing hemi-nested PCR primers for use in real-time PCR, entails PCR primers designed such that the 5′ forward primer is nested within the SCRO, with, for example, 4 bases of the Dengue sequence at the primer's 3′ end to prevent misannealing and extension of the viral genome. The 3′ reverse PCR primer is designed based on the viral genome, as previosuly discussed. Therefore, the amplification of a targeted strand, exemplarily a replicative strand RNA, would be obtained only when RT-PCR is performed using the combination of SCRO primers and the strand-specific hemi-nested PCR primers, with no amplification obtained from random-primed total Dengue RNA, as found in Dengue-infected cells, for example.

Accordingly, the following non-limiting examples are provided in order to further illustrate the teachings of the present invention, here exemplarily applied to nucleic acid strand specific detection and quantification of the replicative form of 2 viruses, the Dengue virus and RSV. In particular examples detection is demonstrated from viruses from in vivo as well as in vitro sources.

In these samples, the following exemplary materials and methods were utilized.

Cell lines and virus: Mosquito cell line C6/36 was maintained in Leibowitz L-15 medium (Invitrogen Life Technologies, Carlsbad, Calif.) supplemented with 10% fetal bovine serum (FBS; Hyclone Laboratories Inc., Logan, Utah) and cultured at 28° C. Baby hamster kidney (BHK-21) cell line was cultured at 37° C. in 5% CO₂, and maintained in RPMI-1640 medium (Sigma, St. Louis, Mo.) supplemented with 10% FBS. The Dengue 2 New Guinea C (NGC) strain virus was propagated in C6/36 cells at 32° C. as described in Gould and Clegg, 1985. The titer of the Dengue 2 virus was evaluated by plaque assay in BHK-21 cells.

Dengue virus infection and titration: Monolayers of C6/36 or BHK-21 cells were grown in 24 well plates and infected the next day with Dengue 2 virus. After adsorption for 1 hour, unbound viruses were removed by aspiration, the cells rinsed with PBS and grown in their respective maintenance media. Cells without Dengue virus infection, or inoculated with heat-inactivated virus (30 minutes at 56° C.), were used as controls. In some experiments, BHK-21 cells were infected with Dengue 2 virus and grown in the absence or presence of varying concentrations of ribavirin (1-fl-D-ribofuranosyl-1,2,4-triazole-3-carboximide; Sigma, St. Louis, Mo.) to inhibit viral transcription. Cytotoxicity of the cells due to ribavirin treatment was determined by CytoTox 96® Non-Radioactive Cytotoxicity Assay (Promega Corporation, Madison, Wis.). The titer of Dengue 2 virus was determined by plaque assay with BHK-21 cells. Briefly, serial dilutions of the virus were adsorbed onto BHK-21 monolayer cells as described above. The viral inoculi were then replaced with RPMI-1640 containing 2% FBS and 1% carboxymethylcellulose (CMC) (Calbiochem®, La Jolla, Calif.). At day 6 post-infection, cells were fixed and stained with 1% crystal violet-4% formalin solution in phosphate-buffered saline (PBS), and the plaque numbers counted. All assays were done in triplicates.

Inoculation of Dengue virus into newborn mice: Intra-cranial (i.c.) inoculation of Dengue 2 virus into newborn Balb/c mice was performed as described (Gould and Clegg, 1985). Litters of newborn mice were inoculated intra-cranially with 1×1 -3 PFU of live or heat-inactivated Dengue 2 virus. The mice were then sacrificed 5 days later and their brains removed, homogenized in TRIzol® Reagent (Invitrogen Life Technologies, Carlsbad, Calif.), and the total RNA prepared according to the manufacturer's instructions. Each RNA sample was analyzed by denaturing gel electrophoresis and quantified by spectrophotometry (21). Three to five ug of total RNA from each brain sample was subsequently used for RT followed by PCR as described below.

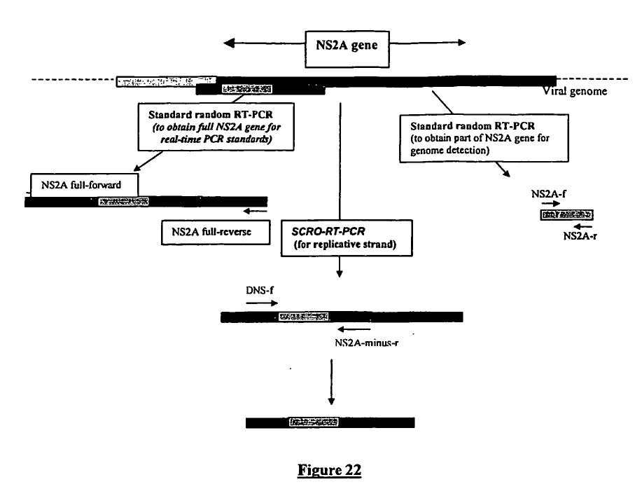

Construction of plasmid and preparation of RNA transcripts by in vitro transcription: The Dengue 2 genomic RNA was extracted as described (Tooet al., 1995) and used as a template for the synthesis of viral cDNA by RT (Sambrook et al., 2001). A schematic of the following example is provided by FIG. 22, depicting the use of various oligos/primers in accordance with the teachings disclosed herein. Briefly, the first strand cDNA was generated by RT at 42° C. for 1 hour using random primers (Promega Corporation, Madison, Wis.). Following RT, the full Dengue 2 NS2A region was amplified by standard PCR using NS2A full-forward (5′-GGACATGGGCAGATTGAC-3′ (SEQ ID NO: 13)) and NS2A full-reverse (5′-TCCTTTCTTGTTGGTTC-3′(SEQ ID NO: 14)) primers, in order to amplify the whole NS2A gene for making PCR standards and as a template for single stranded RNA synthesis. The PCR product was purified using CONCERT™ Rapid PCR Purification System (Invitrogen Life Technologies, Carlsbad, Calif.) and cloned into pGemT (Promega Corporation, Madison, Wis.). The positive clone pGemT-DNS2A, containing the Dengue genomic sequences from nucleotides 3478 to 4132 (Genbank accession no. M29095), was verified by sequencing. The positive and negative RNA strands of Dengue NS2A were then synthesized by in vitro transcription of linearized pGemT-DNS2A using the Riboprobe® in vitro Transcription Systems (Promega Corporation, Madison, Wis.) according to the manufacturer's instructions. The transcribed RNAs were quantified by spectrophotometry and the integrity verified by denaturing gel electrophoresis.

RT-PCR and real-time quantitative PCR of Dengue nucleic acids: Total RNA was prepared from infected cells using the TRizol® Reagent (Invitrogen Life Technologies, Carlsbad, Calif.) according to manufacturer's instructions. The RNA obtained was quantified by spectrophotometry, and their integrity analyzed by gel electrophoresis. The RT reaction was performed at 42° C. on 2-5 ug of total RNA as described (21) using 100 U of M-MLV (Promega Corporation, Madison, Wis.) with either 12.5 pmoles of random hexamers (Promega Corporation, Madison, Wis.) or DNS-1 (5′-TCACCGTTCCCCGCCGTCGGTGGGCGCTAC-3′ (SEQ ID NO: 5)) in a total volume of 10 ul. DNS-1, a sense primer to the negative replicative strand of the Dengue NS2A region, was designed to possess a hairpin stem-loop structure to avoid mispriming at the RT step and unique Dengue sequences to achieve high specificity in amplification step (FIG. 10). The calculated ΔG of the hairpin loop at the RT step was −2.9 kcal/mol, with a Tm (melting temperature) of 62° C. Hence, at the salt and temperature conditions used for RT, the formation of a hairpin loop is highly favoured. However, with the conditions used for PCR, the ΔG and the Tm of the hairpin loop were −0.4 kcal/mol and 64° C. respectively.

Hence at the annealing temperature used in this example (60° C.), the formation of the hairpin loop was less favorable, thus allowing efficient annealing of the PCR primers (DNS2-f and NS2A-minus-r) to the unique sequences. The sequences of the primers used for real-time quantitative PCR are given in Table 2.

TABLE 2

Sequences of primers used in Dengue PCR assays.

| | | Genome | |
|---|---|---|---|
| Gene | Primer sequence | position | (SEQ ID NO:) |
| Env-f | 5′ AGGATGGGGAAATGGATGTGG 3′ | 1233-1253 | (SEQ ID NO: 15) |
| Env-r | 5′ TTCTGTGGCCCCTGTGAGTGC 3′ | 1723-1743 | (SEQ ID NO: 16) |
| NS2A-f | 5′ ACCTGGGAAGAGTGATGGTTATGG 3′ | 3632-3655 | (SEQ ID NO: 17) |
| NS2A-r | 5′ ATGGTCTCTGGTATGGTGCTCTGG 3′ | 3813-3836 | (SEQ ID NO: 18) |
| DNS2-f | 5′ CGTTCCCCGCCGTCGGTG 3′ | | (SEQ ID NO: 19) |
| DNS2A-minus-r | 5′ TCACTGCATTTGGGACGC 3′ | 3938-3955 | (SEQ ID NO: 20) |
| Actin-f | 5′ ACAACGGCTCCGGCATGTGC 3′ | 32-51 | (SEQ ID NO: 21) |
| Actin-r | 5′ GGTCATCTTTTCACGGTTGG 3′ | 41-360 | (SEQ ID NO: 22) |

Sense and antisense primers were denoted with suffixes ‘f’ and ‘r’ respectively. Dengue sequences were assigned in accordance with the Genbank accession number M29095. Actin primers were designed according to sequences reported in Genbank accession number AJ312092.

All real-time quantitative PCR was performed on the iCycler jQ™ Multi-Color Real Time PCR Detection System (Bio-Rad, Hercules, Calif.). The threshold cycles (Ct) were calculated using the Optical interface v2.3B (Bio-Rad, Hercules, Calif.). All standard templates were identical in size and sequence to the targets and were generated by PCR using Taq polymerase (Promega Corporation, Madison, Wis.) and 50 nM of each primer, with the following cycling conditions: a 95° C. step for 3 mm; 25 cycles of 95° C. for 30 s, 55° C. for 30 s, and 72° C. for 1 mm; and an elongation step at 72° C. for 7 mm. All DNA sequences were confirmed by sequencing. Unless stated otherwise, all real-time quantitative PCR was performed with 40 cycles of 30 s denaturation at 95° C., 30 s annealing at 60° C. and 30 s extension at 72° C. Fluorescent detection of SYBR Green I (X-tensaMIX-SG; BioWORKS, Singapore) was carried out at the extension phase. ‘Hot-start’ real-time quantitative PCR was carried out using SYBR® Green PCR Master Mix containing AmpliTaq Gold® DNA polymerase (Applied Biosystem, Foster City, Calif.). The cycling parameters were identical to those described above, with an additional step at 95° C. for 10 mm to activate the AmpliTaq Gold DNA polymerase.

Results

DNA amplification using the SCRO and nested PCR primers, designed to detect the viral negative strand RNA, was specific for cells infected with replicative Dengue virus. The negative RNA strand of the Dengue virus is present in cells only when active viral replication occurs (Chambers et al., 1990). The quantification of this moiety can therefore be used to assess the progression of Dengue replication. The primer DNS-1, designed with a stem-loop structure with unique sequences to prevent misannealing and promote targeted priming (as discussed above) to the negative strand of Dengue NS2A region, was used to specifically reverse transcribe the replicative strand but not the genome of Dengue virus (FIG. 10), where, the primary sequence complementary to the negative replicative strand of Dengue is 5′-GGTGGGCGCTAC-3′ (SEQ ID NO: 23). To assess the specificity of detection, RT-PCR of total RNA obtained from C6/36 cells infected with either replicative or heat-inactivated Dengue 2 virus at a multiplicity of infection (m.o.i.) of 1, was carried out. Total RNA was harvested from each sample when approximately 50% synctia was observed in C6/36 cells infected with the replicative virus. For each sample, an RT reaction was performed using DNS-1, followed by PCR using the primers DNS2-f and NS2A-minus-r (Table 2). The PCR products, when analyzed by gel electrophoresis, showed the expected size of 320 bp with cells infected with the replicative (lanes 1-3) but not with inactivated (lanes 4-6) Dengue virus (FIG. 12). The identities of the PCR products were confirmed by sequencing.

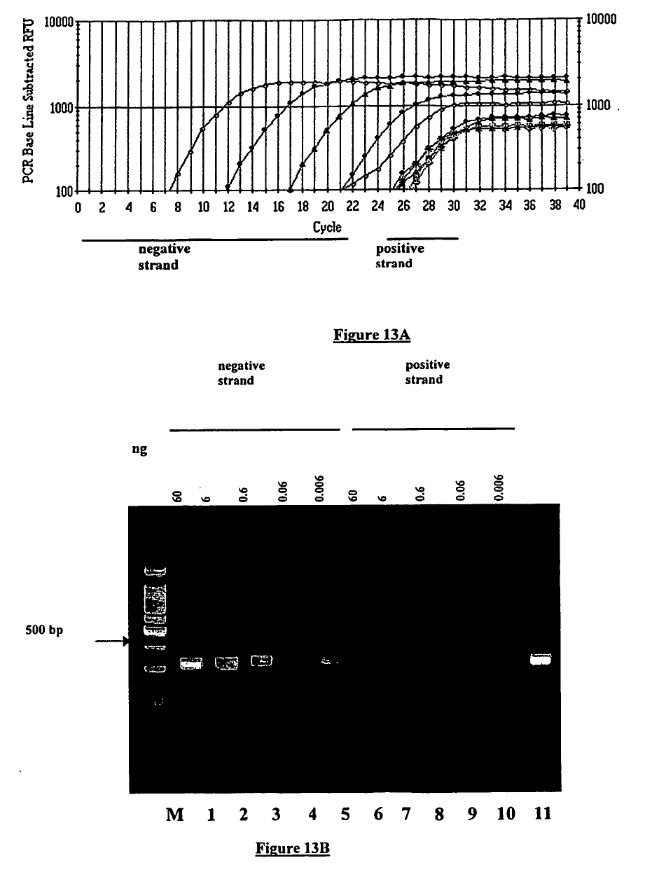

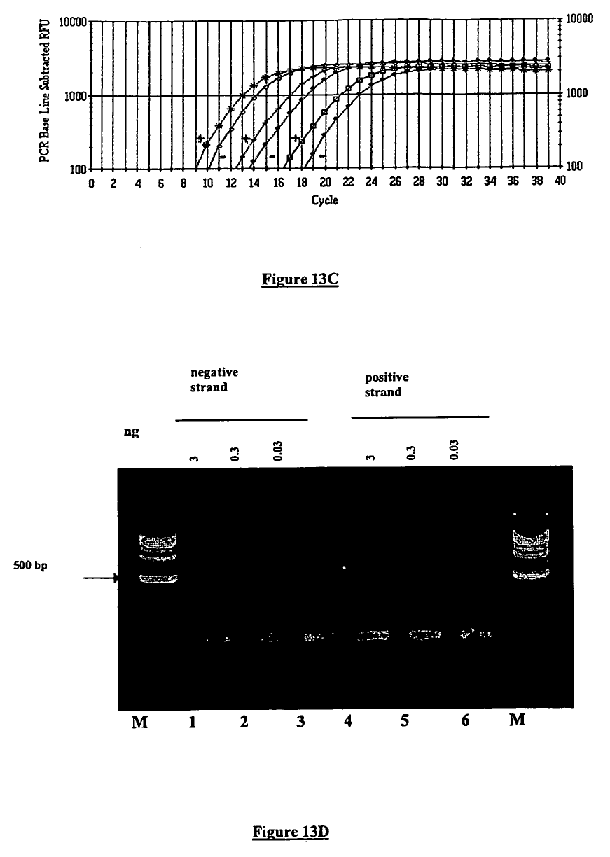

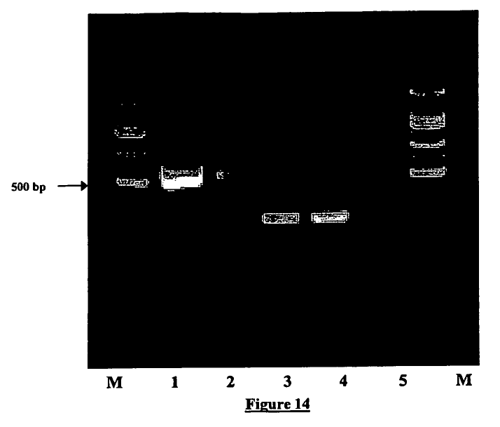

More particularly, FIG. 12 shows RT-PCR analysis of C6/36 cells infected with replicative or inactivated Dengue 2 virus. The mosquito C6/36 cells were infected in triplicates with either replicative (lanes 1-3) or heat-inactivated (lanes 4-6) Dengue 2 virus at an m.o.i of 1. Total RNA from each sample was reversed transcribed with the strand-specific DNS-1 oligonucleotide and amplified with primers DNS2-f and NS2A-minus-r. The expected DNA fragment amplified from the negative RNA strand (315 bp) is indicated (Den). No amplification product was observed from cells exposed to inactivated virus. Lane M, DNA molecular size marker (100 bp ladder). The study was reproduced at least twice with identical results. Primers specific to the negative strand RNA of Dengue virus were unable to amplify the positive strand genomic RNA. The data above indicated that the primers were able to accurately amplify the desired Dengue NS2A region. The specificity of this method to detect Dengue negative RNA strand was further evaluated using two different approaches. Firstly, amplification of serially diluted reverse transcribed strand-specific cDNA was carried out. The positive and negative strand RNAs were synthesized by in vitro transcription using pGemT-DNS2A encoding the Dengue NS2A sequences. Equivalent amounts of the each RNA were reverse transcribed by DNS1 primer and real-time PCR carried out on serially diluted cDNAs using primers DSN2-f and NS2A-minus-r. The negative strand Dengue RNA sample was efficiently detected over the range of concentrations examined (FIG. 13A). However, no specific amplification was observed with any of the positive strand RNA samples, as indicated by the similarities of their Ct values to the control. To validate the observations, gel electrophoresis of the PCR products showed the expected DNA fragments of 320 bp amplified from serially diluted negative strand RNAs (lanes 1-5) but not with the corresponding samples of serially diluted positive strand samples (lanes 6-10) (FIG. 13B). To rule out the possibility of differences in the integrity of the positive and negative strand RNA, both were reverse transcribed with random hexamers and quantified by real-time quantitative PCR using internal Dengue specific primers NS2A-f and NS2A-r (Table 2). Both the positive and negative NS2A RNA strand samples showed comparable Ct values (FIG. 13C) and yielded the expected PCR product sizes of 205 bp (FIG. 13D). The data therefore indicated that this strategy was able to specifically detect the Dengue NS2A negative RNA strand by at least 5 log concentrations over the corresponding positive strand RNA.

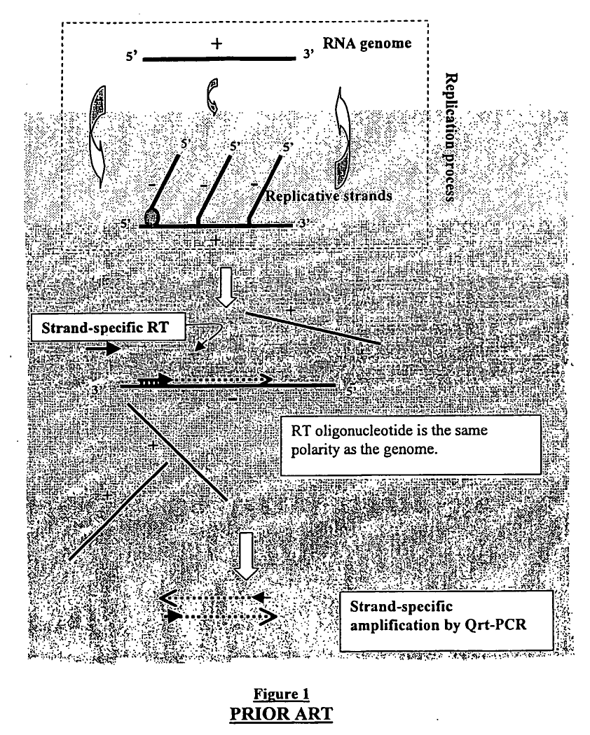

As stated above, FIG. 13 clearly shows specificity and sensitivity of detection of the Dengue negative strand RNA by RT with specific oligonucleotides and real-time PCR. (FIG. 13A) Positive and negative stranded RNA of the Dengue NS2A were synthesized by in-vitro transcription using pGemT-DNS2A as described above. The negative and positive stranded NS2A RNA were reverse transcribed using the DNS-1 oligonucleotide, serially diluted and subsequently detected by real-time quantitative PCR. The underlying bars indicate the relative positions of the curves generated with negative and positive stranded RNA at different dilutions. The negative strand RNA, but not the positive strand, was effectively amplified. The products from the experiment were validated by gel electrophoresis as described in (FIG. 13B). The slopes of the plots of Ct against log of template dilutions of serially diluted negative strand RNA (3.9±0.1) and the corresponding standards (4.3±0.1) were similar. The experiment was repeated as least three times. (FIG. 13B) Gel electrophoresis of the amplified DNA products from the real-time quantitative PCR of Dengue NS2A negative (lanes 1-5) and positive (lanes 6-10) strand RNAs. S, positive control (lane 11) using Dengue NS2A cDNA standard prepared as described above. Note that no amplification product corresponding to the predicted size was obtained with positive strand RNA (lanes 6-10). The apparent amplifications of positive strand Dengue RNA seen in (A) were due to primer-dimer formation (FIG. 1 3C). Validation of the intergrity of the in vitro transcribed negative (−) and positive (+) strand RNAs by real-time quantitative PCR. The RNAs were reverse transcribed from random hexamers, and quantitated using internal Dengue NS2A specific primers, followed by gel electrophoresis analysis (FIG. 13D). All dilutions of either strand RNAs were efficiently amplified. Hence, the failure to detect specific amplification of positive strand RNA using DNS1 during RT in (FIG. 13A) was not due to the differences in the efficiencies of in vitro transcription. M, DNA molecular size marker (100 bp ladder).

A second approach to determine the specificity of this method was the attempt to detect the negative RNA strand directly from the Dengue viral stocks. To verify the strand specificities of the strategy at the RT and amplification steps, random-primed RT was carried out with Dengue genomic RNA extracted directly from viral stocks, followed by real-time PCR using primers directed at the envelope (Env-f and Env-r) or strand-specific primers (DNS2-f and NS2A—minus-r). Random hexamer-primed Dengue genome should be efficiently amplified with non-strand specific primers to the envelope, but not with the pair of strand-specific primers, DNS2-f and NS2A-minus-r. The results indicated that a 511 bp DNA fragment was amplified using primers to the envelope (FIG. 14, lane 2), with no amplification obtained with strand-specific primers (FIG. 14, lane 5), consistent with predicted results. Furthermore, amplification of the negative strand RNA was obtained only when RT-PCR was performed on total RNA from Dengue-infected cells using the combination of DNS-1 and the hemi-nested strand-specific PCR primers, DNS2-f and NS2A-minus-r (lane 4, 320 bp). No amplification was obtained when PCR was performed on random-primed total RNA of infected cells using these hemi-nested strand-specific primers (data not shown). Hence, in accordance with the method taught by the present invention, this method provides specific reverse transcription and amplification of the replicative negative strand Dengue RNA.

As discussed, FIG. 14 depicts results of directed amplification of reverse transcribed Dengue genomic RNA, as enabled by the teachings of the present invention. Specificity of amplification is determined by using random hexamer-primed cDNA prepared from 1.25×10³ PFU of Dengue viral stock. PCR was then performed to detect viral genome (lane 2) and negative strand RNA (lane 5) using the pairs of primers of Env and strand-specific NS2A (DNS2-f and NS2A-minus-r) primers, respectively. The integrity of the strand-specific primers was tested on total cellular RNA of infected cells that had been primed by the DNS1 sense primer (lane 4). Positive controls for the detection of the viral genome (lane 1) and replicative RNA (lane 3) were performed utilizing Dengue Env and NS2A cDNA standards prepared as described previously.

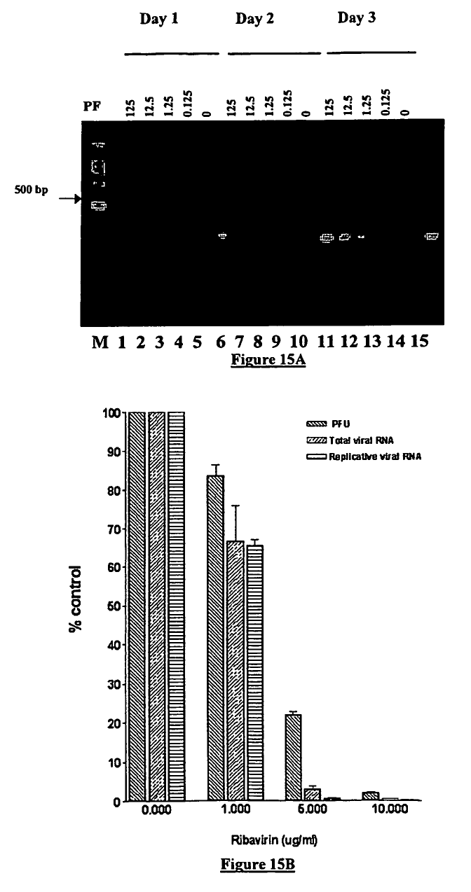

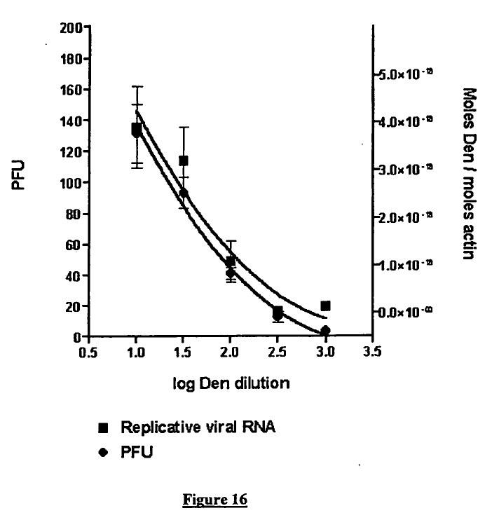

Decrease in replicative negative strand RNA correlated with the plaque-based assay in ribavirin treated BHK-21 cells infected with Dengue: The above studies clearly demonstrated the high specificity and sensitivity in detecting the replicative negative RNA strand of Dengue NS2A. To further validate the utility of this method, a direct measurement was carried out to correlate the levels of replicating virus in infected cells treated with ribavirin. Ribavirin is known to inhibit the replication of many RNA viruses including Dengue (Koff et al., 1982; Sidwell, 1979). The kinetics of Dengue infection of BHK21 cells were initially determined to establish the minimum post-infection time required to produce detectable levels of replicative RNA with a reasonable dynamic range. BHK-21 cells were infected with 10-fold serial dilutions of Dengue virus, and RNA harvested at days 1, 2 and 3 post-infection. BHK-21 cells not infected with the Dengue virus served as negative controls. RT-PCR to detect the replicative negative strand RNA was then performed on equivalent amounts of total cellular RNA. The Dengue negative RNA strand was detected at day 2 post-infection only with an m.o.i of 0.00125 (FIG. 15A, lane 6). On day 3, negative strand RNA was detectable over 4 log dilutions of viruses (FIG. 15A, lanes 11-14). Detection of the Dengue genome with random hexamer-primed RNA samples and primers to the envelope showed similar kinetics (data not shown). Hence, all subsequent studies using ribavirin were carried out at day 3 post-infection.

To correlate the levels of replicating virus and the effects of ribavirin, BHK-21 cells were initially infected with Dengue virus at an m.o.i of 0.00125, in the absence or in the presence of varying concentrations of ribavirin. The supernatants containing extracellular Dengue virus were harvested at day 3 post-infection, and used to infect fresh cultures of BHK-21 cells as described (14), as well as to determine cell toxicity by LDH release. The number of plaques formed (PFU) was then determined by plaque assay, and the relative PFU of the ribavirin treated samples calculated. Ribavirin at the highest concentration used (10 ug/ml) did not induce detectable cell death. Similarly, at 3 days post-infection, no significant cell death (˜2%) was observed with Dengue-infected cells. Ribavirin inhibited viral replication in a concentration dependent manner (FIG. 15B). Random hexamer-primed RT of total RNA and quantification with primers to the envelope region (Table 2) showed a corresponding decrease in viral transcripts. Quantification of the replicative, negative Dengue RNA strand showed a concomitant decrease with increasing concentrations of ribavirin (FIG. 15B). The results further validated the utility of this method to quantify the replicative form of the virus.

Correlation of viral RNA quantification using plaque titration and real-time PCR assays: As real-time quantitative PCR is a robust, rapid and reliable method for diagnosis and quantification, the strategy developed herein is compared to the conventional “gold standard” plaque-based assay. To correlate the quantification of active viruses by real-time PCR with the plaque titration assay, serial dilutions of the Dengue virus were used to infect BHK-21 cells in replicates. Total cellular RNA was then isolated 3 days post-infection from one of these replicates, with the other replicates used for the plaque titration assay. At 3 days post-infection, no significant cell death (˜2%) was observed as measured by LDH release. The data shown in FIG. 16 indicated that the values obtained from the quantification of the replicative Dengue RNA strand were highly correlated to the viral titers.

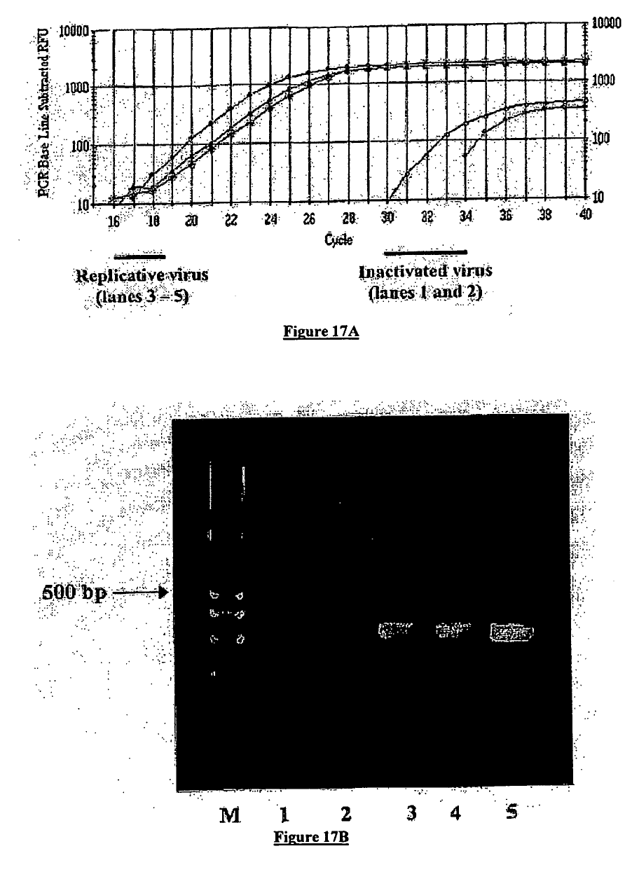

The specific Dengue primers were able to detect and amplify the replicative Dengue virus from the brains of infected mice: To extend the in vitro studies above, this method was used to amplify replicating virus in vivo. Newborn mice were inoculated i.e. with 1×10³ PFU of either replicative or heat-inactivated Dengue virus. Severe symptoms were obvious on day 5 in mice inoculated with the replicative virus. In contrast, mice inoculated with inactivated Dengue virus did not show any detectable physical differences to the controls. Total RNA was then prepared from whole brain, reverse transcribed with DNS-1, and subsequently quantified by real-time quantitative PCR (FIG. 17A). Using identical amounts of RNA from these two groups of mice, RT with DNS-1 and amplification using DNS2-f and NS2A-minus-r primers showed significant differences. Specific amplifications were observed only with samples isolated from brains of mice inoculated with the active (replicative) but not inactivated virus (FIG. 17B). The predicted 320 bp fragment was detected in samples only from brains of mice inoculated with the active virus (lanes 3-5) but not from mice inoculated with the inactivated virus (lanes 1 and 2) when analyzed by gel electrophoresis (FIG. 17B). Amplifications from inactivated virus (FIG. 17A) resulted from primer-dimer formation.

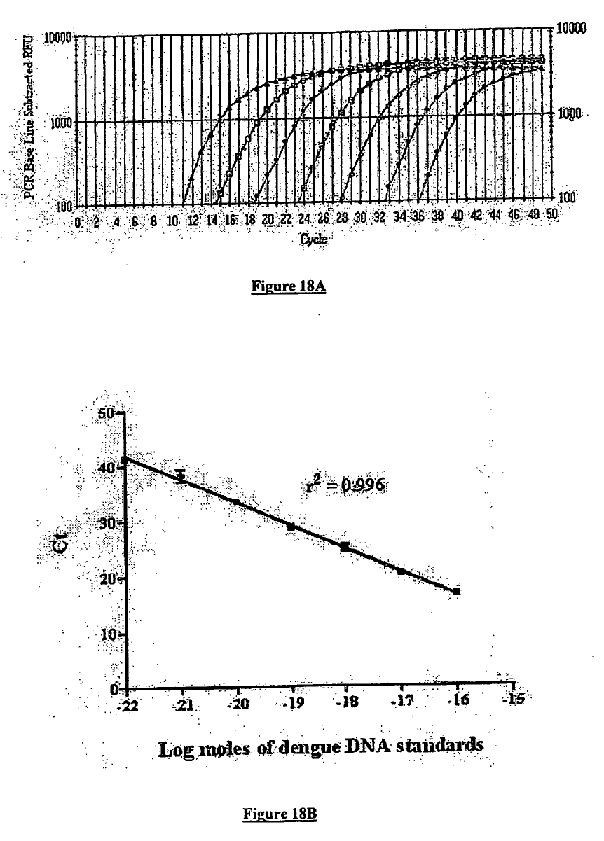

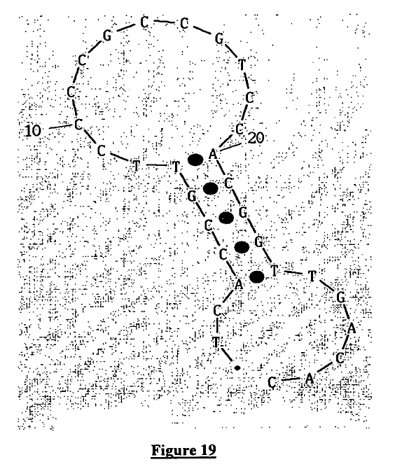

‘Hot-start’ extended the detection limit of real-time PCR using the strand-specific Dengue primers: To explore improvement of the detection limit of real-time quantitative PCR, a ‘hot-start’ strategy was employed. Quantification of serially diluted Dengue DNA standards in the absence of a ‘hot-start’ showed a detection limit of about 10-20 moles, below which primer-dimer formation was detected (FIG. 13C). Using Amplitaq Gold DNA polymerase, the detection was linear over a range of 6 log concentrations of template and a detection limit in the subzeptomoles range, with no formation of primer-dimer (FIG. 18A). The slope of the plot of Ct against log of template concentration (4.18±0.04; FIG. 18B), a showing of the efficiency of amplification of the Dengue fragments using a “hot-start” method, was similar to those obtained without ‘hot-start’ (4.30±0.1). The experiment was repeated at least three times with similar results.

This example exemplifies the simple yet specific and sensitive method taught by the present invention to exclusively detect and quantify the replicative RNA strand of actively replicating Dengue virus. This method is a significant improvement over currently existing strategies reported for the detection of replicating Dengue virus, and offers a potentially useful tool in the quantitative diagnosis of Dengue infection and for studies of Dengue virus tropism in host cells during infection.

The specificity of this new method was achieved at two levels: (1) the design of a SCRO primer containing Dengue and unique sequences, which was folded into a hairpin stem-loop structure with optimal energetics, to avoid mispriming at the RT step (FIG. 13A), and (2) the use of specific nested PCR primers to the SCRO. Using this strategy, the negative strand RNA was detected with a difference of at least five log concentrations over the equivalent positive RNA strand (FIG. 13A). The amplification was specific to negative RNA strand that had been reverse transcribed only by the DNS-1 RT primer, with no amplification observed with random hexamer-primed Dengue cDNA (FIG. 13B). The sensitivity of the method, evaluated with serially diluted Dengue NS2A cDNA standards, was found to be between 100-1000 copy numbers, comparable to previously reported detection method by real-time PCR (Callahan et al. 2001; Houng et al., 2000; Houhg et al., 2001; Laue et al., 1999; Warrilow et al., 2002). The sensitivity of detection was further improved to the subzeptomole range (10 copy number) with the use of a “hot-start” polymerase, with no change in the PCR efficiency (FIG. 17A).

The utility of the method developed herein was further validated by showing a good correlation between the amount of replicative Dengue virus with the number of PFU in infected cells treated with varying concentrations of ribavirin (FIG. 15B). It should be noted that the amount of replicative RNA quantified was lower than the PFU. In evaluating the reduction of PFU due to ribavirin-induced transcription inhibition, media from the treated infected cells were used to infect fresh BHK-21 cells in the absence of ribavirin, a condition that relieved the transcriptional inhibition of newly synthesized viral RNA. A good correlation of the amount of replicative viral RNA was also observed with the conventional plaque-based assay (FIG. 15A). Thus, the combined results indicate the potential use of this method in quantitative diagnosis. A distinct advantage of this strategy over the conventional, labour intensive plaque assay is the significant reduction in time for analyses (6-14 days for a Dengue plaque assay compared to a maximum of 3 days by this method). The use of mosquito cell lines and higher m.o.i. has allowed the detection of replicative intermediates at even shorter time (Lui et al., 1997; Vaughan et al., 2002). However, infections of mosquito cell lines do not allow the quantification by plaque formation. The use of BHK-21 cells in this study enabled the concomitant comparison of the quantitative method described herein with the “gold standard” plaque assay. The use of this method has been extended to in vivo detection of replicating Dengue virus in the brains of infected mice (FIG. 16).

PCR can amplify DNA from non-replicative organisms, producing results that are diagnostically correct as positive, which clinically can be false-positives (Burkardt, 2000). Attempts to detect the replicative forms of the Dengue virus using molecular techniques have met with some success (Lui et al., 1997; Vaughan et al., 2002). Based on the work of Lanciotti et al. to serotype Dengue viruses (Lanciotti et al.,1992), Vaughan and co-workers reported the detection of Dengue RI RNA by conventional RT-PCR. The method was reported to be more rapid than that described by Liu et al., which required Southern blotting for final identification. Using the primers described in their study, resulted in amplifications from D1-, T2- as well as random-primed Dengue RNA from infected cells (unpublished observation) indicative of a lack of strand specificity in the prior art strategy employed. In contrast, the teachings of the present invention provide for amplification of exemplary negative strand RNA, obtained only when RT-PCR was performed using the combination of DNS-1 and the strand-specific PCR primers (DNS2-f and NS2A-minus-r), with no amplification obtained from random-primed total RNA of Dengue-infected cells. Furthermore, the approaches of both Liu et al. and Vaughan et al. were not quantitative in detecting replicative Dengue virus. The teachings of the present invention provide for both quantitative and exclusive detecting of replicative RNA strand, a marked improvement over previosuly known methods.

Hence, the availability of this method to specifically detect Dengue replicative strand RNA will enable the study of Dengue virus tropism towards host cells during infection. In accordance with the teachings of the present invention, the method developed herein for the detection of replicative Dengue virus can be extended to the studies of other viruses. This approach has successfully been applied to the quantification of the replicative form of another flavivirus, the West Nile virus, in both infected cells and mice

In another embodiment, the teachings of the present invention were also utilized to detect the positive replicative strand of another exemplary virus, namely RSV. The appropriate RT and PCR primers were designed based on the criteria stated above to detect the replicative positive strand of the RSV. The specificity and sensitivity of these primers were then assessed by the same format as the Dengue virus.

RSV specific primers, targeted to the viral positive strand RNA, were able to detect and amplify the RSV-specific MP2 region: The positive RNA strand of the RSV is present in cells only when active viral replication occurs. Quantitation of this moiety can therefore be used to assess the progression of replication. The primer RSVRT1, designed according to the thermodynamic and sequence selection parameters taught herein, to prevent misannealing and promote targeted priming to the positive strand of RSV MP2 region, was used to specifically reverse transcribe the replicative strand but not the genome of RSV virus (FIG. 19; SEQ ID NO: 29).

FIG. 19 depicts another exemplary SCRO designed in accordance with the teachings of the present invention. Primary sequence and predicted secondary structure of RSVRT1 oligonucleotide is shown. The folding algorithm, designed by D. Stewart and M. Zucker, was used to generate thermodynamic parameters (http://bioinfo.math.rpi.edu/˜zukerm/). The primary sequence complementary to the positive replicative strand is 5′-CACGGTGACAC-3′ (SEQ ID NO: 24).

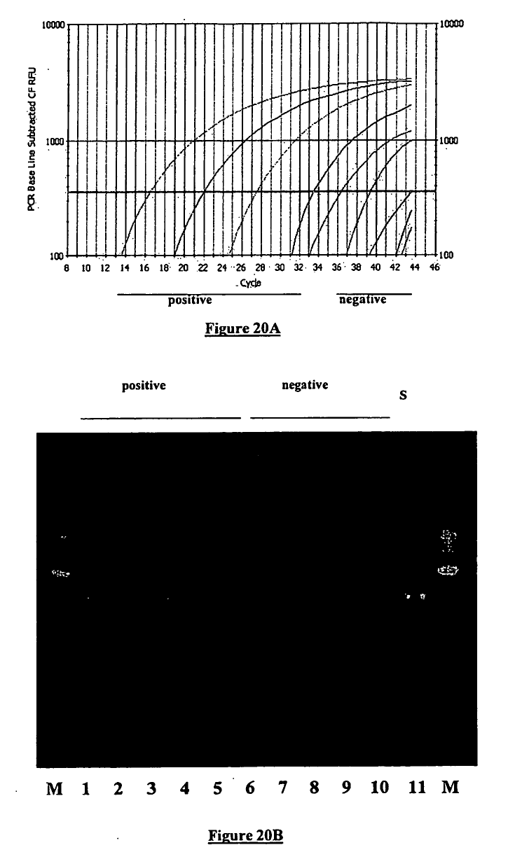

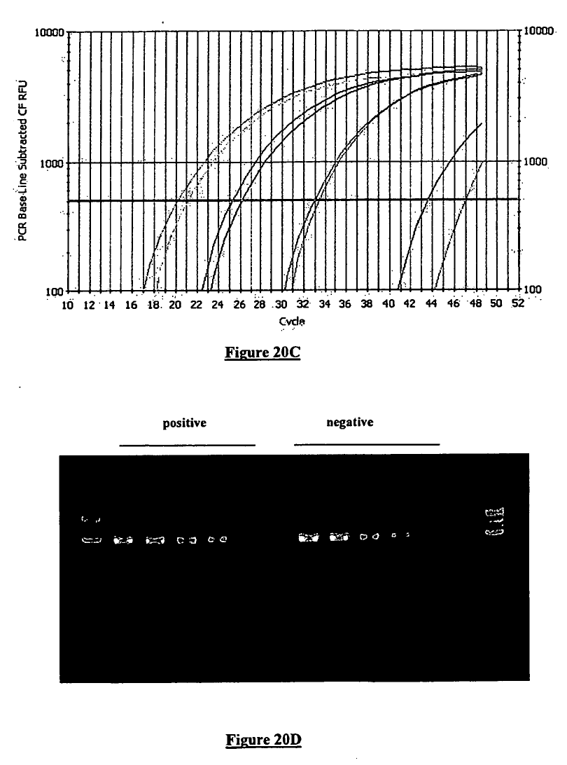

Unique RSV primers, designed in accordance with the teachings of the present invention, exclusively detected and amplified the positive strand RNA of the RSV virus with high specificity and sensitivity. The specificity of this method to detect RSV positive strand RNA was evaluated using two different approaches. Firstly, amplification of serially diluted reverse transcribed strand-specific cDNA was carried out. The positive and negative strand RNAs were synthesized by in vitro transcription using pGemT-RSVMP2 encoding the matrix protein 2 (MP2) DNA sequences. Equivalent serially diluted amounts of the each RNA were reverse transcribed and real-time PCR carried out using primers RSVS and RSVnAS (Table 3). The positive strand RSV RNA sample was efficiently detected over the range of concentrations examined (FIG. 20A). However, no specific amplification was observed with any of the genome strand RNA samples as indicated by the similarities of the Ct values to the control. To validate the observations, gel electrophoresis of the PCR products showed the expected DNA fragments of 320 bp with serially diluted positive strand RNAs (lanes 1-5) but not with the corresponding samples of serially diluted negative strand samples (lanes 6-10) (FIG. 20B).

FIG. 20A depicts schematically the specificity and sensitivity of detection of the RSV positive strand RNA by RT with SCROs and real-time PCR. The negative and positive stranded MP2 RNA were serially diluted, reverse transcribed using the RSVRT1 oligonucleotide, and subsequently detected by real-time quantitative PCR. The underlying bars indicate the relative positions of the curves generated with negative and positive stranded RNA at different dilutions. The positive strand RNA, but not the negative strand, was effectively amplified. Products from the experiment were validated by gel electrophoresis as described previously. S; standard.

To rule out the possibility of differences in the integrities of the positive and negative strand RNA, both were reverse transcribed with random hexamers and quantitated by real-time quantitative PCR using RSV MP2-specific primers RSVS and RSVMP2AS (Table 3). The sequences of the primers used are shown in Table 2. Both the positive and negative MP2 RNA strand samples showed comparable Ct values (FIG. 20C) and yielded the expected PCR product sizes of 559 bp (FIG. 20C).

TABLE 3

Sequences of primers used in RSV PCR assays.

| | | Genome | |
|---|---|---|---|
| Gene | Primer sequence | position | (SEQ ID NO:) |
| RSVS | 5′ CTCTTGGTATAGTTGGAGTGC 3′ | 7834-1253 | (SEQ ID NO: 25) |
| RSVnAS | 5′ TCACCGTTCCCCGCCGTCCAC 3′ | 8149-8151 | (SEQ ID NO: 26) |
| RSVMP2AS | 5′ TTGGAGAAATTGTTGAGTGGC 3′ | 8372-8392 | (SEQ ID NO: 27) |
| Actin-f | 5′ ACAACGGCTCCGGCATGTGC 3′ | 32-51 | (SEQ ID NO: 21) |
| Actin-r | 5′ GGTCATCTTTTCACGGTTGG 3′ | 41-360 | (SEQ ID NO: 22) |

Sense and antisense primers were denoted with suffixes ‘S’ and ‘AS’ respectively. RSV sequences were assigned in accordance with the Genbank accession number AF013254. Actin primers were designed according to sequences reported in Genbank accession number AJ312092. The sequences of the primers used in this study (Table 3) provide an example of a strategy, in accordance with the teachings of the invention, that provides for detection of the RSV replicative positive strand RNA by at least 5 logs over the genome strand.

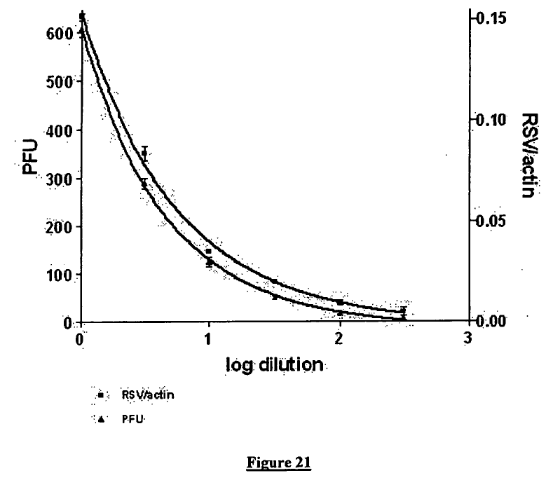

Correlation of viral RNA quantitation using plaque titration and real-time PCR assays: As real-time quantitative PCR is a robust, rapid and reliable for diagnosis and quantitation, the method developed herein is compared to the conventional “gold standard” plaque-based assay. To correlate the quantitation of active viruses by real-time PCR with the plaque titration assay, serial dilutions of the RSV virus were used to infect Vero cells in duplicates. Total cellular RNA was then isolated 6 days post-infection from one of these replicates and the other replicate was used for the plaque titration assay. At 6 days post infection, no significant cell death (<2%) was observed as measured by LDH release. The data shown in FIG. 21 indicated that the values obtained from the quantitation of the replicative RSV RNA strand were highly correlated to the viral titres. The results are expressed as mean ±SD and the experiment was repeated at least three times.

The teachings of the present invention can be extended to the quantification of replicative states of any RNA viruses by distinguishing and detecting the RNA moiety that is indicative of the replication state, as well as to other nucleic acid strands. This approach has successfully been applied to the quantification of the replicative form of both the positive and negative strand RNA viruses.

While the exemplary detection method used to detect the amplified replicative RNA in this strategy uses real-time PCR (thermocycling), isothermic amplification methods e.g., NASBA, rolling circle etc., can be utilized in accordance with teachings described herein and can be modified for each format accordingly by one of ordinary skill in the art.

Furthermore, a plurality of replicative viruses can be detected simultaneously by multiplexing. SCROs for each target can be designed and specific multiplex FRET fluoroprobes can be in used to detect gene specific sequences of the amplified product.

The use of SCROs described herein to detect the replicative RNA is exemplarily performed in a homogenous liquid phase. SCRO can also be immobilized onto a solid phase, keeping all the necessary characteristics for specificity intact, and used in RT for detection and transcription of the replicative RNA, or any other suitable nucleic acid strand of interest.

References to various works have been cited herein and all are incorporated by reference in their entirety as if each work had been incorporated by reference individually.

Although the present invention has been described in connection with the preferred form of practicing it, those of ordinary skill in the art will understand that many modifications can be made thereto without departing from the spirit of the present invention. Accordingly, it is not intended that the scope of the invention in any way be limited by the above description.

### REFERENCES

Bielefeldt-Obmann, H. 2000. Measuring virulence without a target. Trends Microbiol. 8: 265-266.
Burkardt, H. J. (2000) Standardization and quality control of PCR analyses. Clin. Chem. Lab. Med., 38, 87-91.
Callahan, J. D., S. J. Vu, A. Dion-Schultz, B. E. Mangold, L. F. Peruski, D. M. Watts, K. R. Porter, G. R. Murphy, W. Suharyono, C. C. King, C. G. Hayes, and J. J. Temenak. 2001. Development and evaluation of serotype- and group-specific fluorogenic reverse transcriptase PCR (TaqMan) assays for Dengue virus. J. Clin. Microbiol. 39: 4119-4124.
Chambers, T. J., Hahn, C. S., Galler. and Rice, C. M. (1990) Flavivirus genome organization, expression and replication. Annu. Rev. Microbiol., 44, 649-688.
Chu, P. W. and Westaway, E. G. (1985) Replication strategy of Kunjin virus: evidence for recycling role of replicative form RNA as template in semiconservative and asyrnmetric replication. Virology, 140, 68-79.
Cleaves, G. R. Ryan, T. E. and Schlesinger, R. W. (1981) Identification and characterization of type 2 Dengue virus replicative intermediate and replicative forms RNA. Virology, 111, 73-80.
Craggs, J. K., Ball, J. K., Thomson, B. J., Irving, W. L. and Grabowska, A. M. (2001) Development of a strand-specific RT-PCR based assay to detect the replicative form of hepatitis C virus RNA. J. Virol. Methods., 94, 111-120.
Dorries, R (2001) The role of T-cell-mediated mechanisms in virus infections of the nervous system. Curr. Top. Microbiol. Immunol., 253, 219-45.
Deubel, V. 1997. The contribution of molecular techniques to the diagnosis of Dengue infection, p. 335-365. In D. J. Gubler and G. Kuno (ed.), Dengue and Dengue Hemorrhagic Fever, CAB International, New York
Ginzinger, D. G. (2002) Gene quantification using real-time quantitative PCR: An emerging technology hits the mainstream. Exp. Hematology, 30, 503-512.
Gould, E. A., and J. C. S. Clegg. 1985. Growth, titration and purification of alphaviruses and flaviviruses, p. 43-78. In B. W. J. Mahy (ed.), Virology: A Practical Approach, IRL Press, Oxford.
Gubler, D. J. 2002. Epidemic Dengue/Dengue hemorrhagic fever as a public health, social and economic problem in the 21st century. Trends Microbiol. 10: 100-103.
Gunji, T., Kato, N., Hijikata, M., Hayashi, K., Siatoh, S., Shimotohno, K. (1994) Specific detection of positive and negative stranded hepatitis C viral RNA using chemical RNA modification. Arch. Virol. 134, 293-302.
Guzrnan, M. G., and G. Kouri. 2002. Dengue: an update. Lancet Infect. Dis. 2: 33-42.
Houng, H. S., D. Hritz, and N. Kanesa-thasan. 2000. Quantitative detection of Dengue 2 virus using fluorogenic RT-PCR based on 3′-noncoding sequence. J. Virol. Methods 86: 1-11.
Houng, H. S., R. Chung-Ming Chen, D. W. Vaughn, and N. Kanesa-thasan. 2001. Development of a fluorogenic RT-PCR system for quantitative identification of Dengue virus serotypes 1-4 using conserved and serotype-specific 3′ noncoding sequences. J. Virol. Methods 95: 19-32.
Isturiz, R. E., D. J. Gubler, and J. Brea del Castillo. 2000. Dengue and Dengue hemorrhagic fever in Latin America and the Caribbean. Infect. Dis. Clin. North Am. 14: 121-140.
Koff, W. C., Elm, J. L. and Halstead, S. B. (1982) Antiviral effects of ribavirin and 6-mercapto-9-tetrahydro-2-furylpurine against Dengue viruses in vitro. Antiviral Res., 2, 69-79.
Kuno, G., D. J. Gubler, M. Velez, and A. Oliver. 1985. Comparative sensitivity of three mosquito cell lines for isolation of Dengue viruses. Bull. World Health Organ. 63: 279-286.
Lanciotti, R. S., C. H. Calisher, D. J. Gubler, G. J. Chang, and A. V. Vomdam. 1992. Rapid detection and typing of Dengue viruses from clinical samples by using reverse transcriptase-polymerase chain reaction. J. Clin. Microbiol. 30: 545-551.
Lanford, R. E., Sureau, C., Jacob, J. R., White, R., Fuerst, T. R. (1994) Demostration of in vitro infection of chimpanzee hepatocytes with hepatitis C virus using strand-specific RT/PCR. Virol. 202, 606-614.
Laskus, T., Radkowski, M., Wang, L. F., Cianciara, J., Vargas, H., and Rakela, J. (1997) Hepaitis C virus negative strand is not detected in perpheral blood mononuclear cells and viral sequences are identical to those in serum: a case against extrahepatic replication. J Gen. Virol. 78, 2747-2750.
Laue, T., P. Emmerich, and H. Schmitz. 1999. Detection of Dengue virus RNA in patients after primary or secondary Dengue infection by using the TaqMan automated amplification system. J. Clin. Microbiol. 37: 2543-2547.
Lerat, H., Berby, F., Traband, M. A., Vidalin, O., Major, M., Trepo, C., and Inchauspe, G. (1996) Specific detection of Hepatitis C minus strand RNA in hemotopoietic cells. J. Clin. Invest. 97, 845-851.
Lin, L., Fevery, J. and Yap, S. H. (2002) A novel strand-specific RT-PCR for detection of hepatitis C virus negative-strand RNA (replicative intermediate): evidence of absence or very low level of HCV replication in peripheral blood mononuclear cells. J. Virol. Methods., 100, 97-105.
Liu, H. S., Lin, Y. L. and Chen, C. C. (1997) Comparison of various methods of detection of different forms of Dengue virus type 2 RNA in cultured cells. Act. Virol., 41, 317-324
Mellor, J., Haydon, G., Blair, C., Livingstone, W., and Simmons, P. (1998) Low level or absent in vivo replication of hepatitis C virus and hepatits G/GB virus C in peripheral blood mononuclear cells. J. Gen. Virol. 79, 705-714.
Mizutani, T., Kato, N., Ikeda, M., Sugiyama, K., Shimotohno, K. (1996) Long-term human T-cell culture system supporting hepatitis C virus replication. Biochem. Biophys. Res. Comm. 227, 822-826
Navas, M. C., Fuchs, A., Schvoerer, E., Bohbot, A., Aubertin, A. M. and Stoll-Keller, F. (2002) Dendritic cell susceptibility to hepatitis C virus genotype 1 infection. J Med Virol., 67, 152-61
Monath, T. P. 1994. Dengue: the risk to developed and developing countries.
Rosen, L., and D. J. Gubler. 1974. The use of mosquitoes propagate Dengue viruses. Am. J. Trop. Med. Hyg. 23: 1153-1160; Proc. Natl. Acad. Sci. USA. 91: 2395-2400.
Sambrook, J., and D. W. Russell. 2001. Molecular Cloning:Manual, 3rd ed. CSHL Press, Cold Spring Harbor, New York.
Seipp, S., Goeser, T., Theilmann, L. and Kallinowski, B. (1998) Establishment of a highly specific detection system for GB virus C (BGV-C) minus-strand RNA. Vir. Res., 56, 183-189.
Sidwell, R. W., Robbins, R. K. and Hillyard, I. W. (1979) Ribavirin: an antiviral agent. Pharmacol. Ther., 6, 123-146.
Too, H. P. and Maggio, J. E. (1995) Simultaneous extraction of total RNA and peptides from tissues: application to tachykinins. Peptides., 16,45-53.
Too, H P & Wong Y W. “Quantification of alternatively spliced GFRalpha 2 isoforms by sequence-independent detection Real Time PCR”. 10^{th} Queenstown Molecular Biology Meeting. 13-18 Aug. 2000. p 26. Proceedings.
Too H. P. “Real time PCR quantification of GFRα-2 alternatively spliced isoforms in murine brain and peripheral tissues”. Brain Res Mol Brain Res. 10; 114(2): 146-53, 2003
Vaughan, G., Olivera, H., Santos-Argumedo, L., Landa, A., Briseno, B. and Escobar-Gutierrez,A. (2002) Dengue virus replicative intermediate RNA detection by reverse-transcription-PCR. Clin. Diagn. Lab. Immunol., 9, 198-200.
Vorndam, V. and G. Kuno. 1997. Laboratory diagnosis of Dengue virus infections, p. 313-333. In D. J. Gubler and G. Kuno (ed.), Dengue and Dengue Hemorrhagic Fever, CAB International, New York.
Wang, W. K., Sung, T. L., Tsai, Y. C., Kao, C. L., Chang, S. M., and King, C. C. (2002) Detection of Dengue virus replication in peripheral blood minoncleat cells from Dengue virus type 2-infected patients by reverse transcription-real time PCR assay. J. Clin. Micro., 40, 4472-4478.
Warrilow, D., J A. Northill, A. Pyke, and G. A. Smith. 2002. Single rapid TaqMan fluorogenic probe based PCR assay that detects all four Dengue serotypes. J. Med. Virol. 66: 524-528.
Woodall, J. P., C. G. Moore, G. E. Sather, and G. Kuno. 1979. The laboratory diagnosis of Dengue by the mosquito inoculation technique. Pan American Health Organization Scientific Publication 35: 159-164.
Wong, Y. W. , Sia G. M. & H. P. Too. “Quantification of mouse glial cell-line derived neurotrophic factor family receptor alpha 2 alternatively spliced isoforms by real time detection PCR using SYBR Green I”. Neurosci Lett. 8;320(3):141-5, 2002.
Zuker, M. (2000) Calculating nucleic acid secondary structure. Curr. Opin. Struct. Biol., 10, 303-310.

1. A method for strand specific amplification, comprising:
determining nucleic acid sequences of a target nucleic acid strand;
designing a convertible oligonucleotide based, at least in part, on said target nucleic acid strand;
conducting a transcription reaction utilizing said convertible oligonucleotide and said target nucleic acid strand to provide at least one resultant complementary strand;
conducting an amplification reaction to amplify said at least one resultant complementary strand; and
analyzing said amplification reaction.

2. The method of claim 1, wherein said designing step further comprises a step of conducting thermodynamic analysis of said convertible oligonucleotide to predict secondary structure of said convertible oligonucleotide under reaction conditions of at least one of said transcription reaction and under said amplification reaction.

3. The method of claim 2, wherein said predicted secondary structure of said convertible oligonucleotide provides for at least a portion thereof in a stem-loop conformation under conditions of said transcription reaction.

4. The method of claim I, wherein said designing step further comprises the step of considering predicted secondary structures of at least a first portion and a second portion of said convertible oligonucleotide, under differing reaction conditions.

5. The method of claim 1, wherein said designing step includes selecting nucleotides to provide said convertible oligonucleotide with at least a step-loop portion and a portion for annealing to at least a portion of said target nucleic acid strand.

6. The method of claim I, further comprising the step of providing at least one hemi-nested primer for use in said amplification reaction.

7. The method of claim 6, wherein said at least one hemi-nested primer has a Ta that is substantially similar a Tm of said a convertible oligonucleotide, under reaction conditions of said amplification reaction.

8. The method of claim 6, wherein said hemi-nested primer includes a 3′ portion having added nucleotides complementary to a transcription reaction product that is itself at least in part complementary to said target sequence.

9. The method of claim 1, wherein said designing step includes designing said a convertible oligonucleotide which comprises nucleotides that are complementary to said target nucleic acid strand and non-complementary portions to said target nucleic acid strand, wherein said non-complementary portions form a first conformation structure under said transcription reaction conditions and wherein said same non-complementary portions form a second conformation structure under said amplification reactions.

10. The method of claim 9, wherein said first conformation structure has at least a stem-loop portion.

11. A convertible oligonucleotide, comprising:
a first self-annealing portion; and
a second portion complementary, at least in part, to a target nucleic acid sequence.

12. The convertible oligonucleotide of claim 11, wherein said first self annealing portion is in a stem-loop conformation under conditions of a first reaction and converts to a second substantially linear conformation under differing reaction conditions than said first reaction conditions.

13. The convertible oligonucleotide of claim 1 1, wherein said second portion is from about 5 about 15 nucleotides or from about 8 to about 12 nucleotides.

14. The convertible oligonucleotide of claim 12, wherein said convertible oligonucleotide content of guanine, cytosine or combination of both is equal to or greater than 50% of the total nucleotide composition.

15. The convertible oligonucleotide of claim 12, wherein said first self-annealing portion has a ΔG of about ≦−0.5 kcal/mol under transcription reaction conditions.

16. The convertible oligonucleotide 12, wherein said differing reaction conditions are at least transcription and amplification reaction conditions.

17. A method for designing multi-conformational chimeric nucleotides and hemi-nested primers, comprising:
designing a stem-loop chimeric reverse transcription oligonucleotide having two segments, a 5′ stem-loop segment and a 3′ target annealing segment
conducting a reverse transcription reaction utilizing said stem-loop chimeric reverse transcription oligonucleotide and a target nucleic acid sequence, wherein said stem-loop chimeric reverse transcription oligonucleotide's 3′ target annealing segment anneals to said target nucleic acid sequence and said 5′ stem-loop segment forms a self annealing stem-loop structure during said reverse transcription reaction;
subjecting reaction products of said reverse transcription reaction to an amplification reaction wherein nucleic acid sequences of said stem-loop chimeric reverse transcription oligonucleotide adopt a secondary structure sufficient to allow for hybridization of a hemi-nested primer for use in said amplification reaction; and
analyzing results obtained from said amplification reaction.

18. The method of claim 17, further comprising the step of calculating said stem-loop segment's free energy and Tm.

19. The method of claim 17, wherein said 3′ segment's melting temperature is about ±7 degrees centigrade of said reverse transcription reaction's temperature.

20. The method of claim 17, wherein said 3′ target annealing segment has a AG greater than or equal to about −0.5 kcal/mole under said reverse transcription reaction conditions.

21. The method of claim 17, wherein said 5′ stem-loop segment has a ΔG less than or equal to about −0.5 kcal/mole under said reverse transcription reaction conditions.

22. A stem-loop chimeric oligonucleotide, comprising;
a first portion capable of forming a self-annealing stem loop under a first set reaction conditions;
a second portion that maintains a substantially linear conformation under said same first set of reaction conditions and is capable of annealing to a target nucleic acid sequence on a particular strand of nucleic acid.

23. The stem-loop chimeric oligonucleotide of claim 22, wherein said first portion capable of forming a self-annealing stem loop and said second portion have appropriate nucleotide sequences to form a substantially linear confirmation at a second set of reaction conditions dissimilar to said first set of reaction conditions.

24. A stem-loop chimeric oligonucleotide of claim 22 or 23, wherein said first set of reaction conditions are transcription reaction conditions and said second set of reaction conditions are amplification reaction conditions.
